(19) United States
(12) Patent Application Publication
(10) Pub. No.: US 2017/0056479 A1
(43) Pub. Date: Mar. 2, 2017

(54) EXTRACELLULAR MATRIX COMPOSITIONS

(71) Applicant: ANTHROGENESIS CORPORATION, Warren, NJ (US)

(72) Inventors: Mohit B. BHATIA, Manalapan, NJ (US); Uri HERZBERG, Bridgewater, NJ (US); Aleksandr KAPLUNOVSKY, Budd Lake, NJ (US); Raihana ZAKA, Randolph, NJ (US); Qian Ye, Livingston, NJ (US)

(21) Appl. No.: 15/340,924

(22) Filed: Nov. 1, 2016

Related U.S. Application Data
(63) Continuation of application No. PCT/US2015/046690, filed on Aug. 25, 2015.
(60) Provisional application No. 62/041,468, filed on Aug. 25, 2014.

(51) Int. Cl. A61K 38/39; A61K 9/70; A61K 35/50

(52) U.S. Cl. CPC A61K 38/39; A61K 35/50; A61K 9/70

(57) ABSTRACT

Provided herein are placental extracellular matrix (ECM) compositions and methods of making the same. Also provided herein are uses of the placental ECM compositions provided herein.

This application claims priority to U.S. Provisional Patent Application No. 62/041,468, filed Aug. 25, 2014, the disclosure of which is incorporated herein by reference in its entirety.

### 1. FIELD

Provided herein are extracellular matrix (ECM) compositions and methods of making the same. Also provided herein are uses of the ECM compositions provided herein.

### 2. BACKGROUND

Extracellular matrix (ECM) comprises proteins that form many structures in the body, including tendons, ligaments, and sheets that support skin and internal organs. There remains a need in the art for new and improved ECM compositions and methods of making such ECM compositions.

### 3. SUMMARY

In one aspect, provided herein are extracellular matrix (ECM) compositions prepared using placental tissue, e.g., human placental tissue.

In certain embodiments, the ECM compositions provided herein comprise about 30% to about 60% collagen and about 10% to about 35% elastin. In addition, such ECM compositions comprise (i) very low amounts of fibronectin (e.g., less than 0.1% fibronectin), e.g., as measured by ELISA; (ii) no or an undetectable amount of laminin, e.g., as measured by ELISA; and/or no or an undetectable amount of glycosaminoglycans, e.g., as measured by ELISA.

In certain embodiments, the ECM compositions provided herein comprise about 30% to about 72% collagen and about 10% to about 35% elastin. In addition, such ECM compositions can comprise (i) fibronectin (e.g., less than 0.1% fibronectin), e.g., as measured by ELISA; (ii) laminin (e.g., less than 0.1% laminin), e.g., as measured by ELISA; glycosaminoglycans, (e.g., less than 0.1% glycosaminoglycans) e.g., as measured by ELISA; (iii) no or an undetectable amount of cytokines; (iv) no or an undetectable amount of growth factors; and/or (v) no or an undetectable amount of deoxycholic acid.

The ECM compositions provided herein comprise characteristics that make them well-suited for therapeutic/medical use. Specifically, the ECM compositions described herein are sterile, acellular (e.g., ≧99% cell-free) and/or are free of cellular debris (e.g., ≧99% free of cellular debris). In particular embodiments, the ECM compositions provided herein comprise no cytokines or an undetectable amount of cytokines, as measured by, e.g., ELISA. In certain embodiments, the ECM compositions provided herein further are devoid of reagent residuals, i.e., the final ECM compositions comprise undetectable amounts of reagents used in the manufacture of the compositions. Further, in particular embodiments, the ECM compositions provided herein comprise minimal amounts of nucleic acid (e.g., ˜41-171 ng/mg of dry product) and endotoxin (e.g., <0.25 EU).

Another advantageous characteristic of the ECM compositions provided herein is their ability to absorb water. In certain embodiments, the ECM compositions provided herein absorb between 150%-225% their weight in water. Such a characteristic is advantageous in, e.g., wound healing applications of the ECM compositions provided herein.

In a specific embodiment provided herein is an ECM composition comprising about 35-55% collagen and about 10-30% elastin. In a specific embodiment, said composition comprises 34-53% collagen and 13-29% elastin. In another specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In a specific embodiment provided herein is an ECM composition comprising about 35-72% collagen and about 15-25% elastin. In another specific embodiment provided herein is an ECM composition comprising about 40-70% collagen and about 15-25% elastin. In another specific embodiment, said composition comprises 40-70% collagen and 15-22% elastin. In yet another specific embodiment, said composition comprises 43-68% collagen and 18-21% elastin. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin. In another specific embodiment, said ECM composition comprises laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin. In another specific embodiment, said ECM composition comprises glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans. In another specific embodiment, said ECM composition comprises no or an undetectable amount of cytokines, growth factors, and/or deoxycholic acid. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin, laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin, glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans, and, additionally, comprises no or an undetectable amount of cytokines, growth factors, and/or deoxycholic acid. In another specific embodiment, said ECM composition is obtained from placental tissue, e.g., human placental tissue. In another specific embodiment, said ECM composition is obtained from the chorion of a placenta, for example the chorionic plate of a placenta, e.g., the chorion, for example the chorionic plate, from a human placenta.

In another specific embodiment provided herein is an ECM composition comprising about 50-60% collagen and about 10-20% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 45-55% collagen and about 15-25% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 40-50% collagen and about 20-30% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 30-40% collagen and about 25-35% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 34-43% collagen and about 16-24% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 37-42% collagen and about 16-24% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 30-70%, 30-60%, 30-50%, 30-40%, 30-35%, 34-43%, 35-72%, 35-40%, 37-42%, 40-70%, 40-60%, 40-50%, 40-45%, 40-65%, 43-68%, 45-50%, 50-55%, or 55-60% collagen and about 10-30%, 10-20%, 10-15%, 15-25%, 15-22%, 15-20%, 16-24%, 17-24%, 18-21%, 18-20%, 20-24%, 20-30%, 20-25%, 25-30%, or about 30-35% elastin. In a specific embodiment, said ECM composition is obtained from placental tissue, e.g., human placental tissue. In another specific embodiment, said ECM composition is obtained from the chorion of a placenta, for example the chorionic plate, e.g., the chorion, for example, the chorionic plate, from a human placenta. In another specific embodiment, said ECM composition comprises a very low amount of fibronectin, e.g., less than 0.1% fibronectin, less than 0.05% fibronectin, less than 0.01% fibronectin, or less than 0.001% fibronectin. In certain embodiments, said ECM composition comprises 0.001 to 0.1% fibronectin, 0.001 to 0.05% fibronectin, 0.001 to 0.01% fibronectin, 0.01 to 0.1% fibronectin, or 0.01 to 0.05% fibronectin. In another specific embodiment, said ECM composition comprises no or an undetectable amount of laminin. In another specific embodiment, said ECM composition comprises a very low amount of laminin, e.g., less than 0.1% laminin, less than 0.05% laminin, less than 0.01% laminin, or less than 0.001% laminin. In another specific embodiment, said ECM composition comprises laminin, e.g., 0.001 to 0.1% laminin, 0.001 to 0.05% laminin, 0.001 to 0.01% laminin, 0.01 to 0.1% laminin, or 0.01 to 0.05% laminin. In another specific embodiment, said ECM composition comprises no or an undetectable amount of glycosaminoglycans. In another specific embodiment, said ECM composition comprises a very low amount of glycosaminoglycans, e.g., less than 0.1% glycosaminoglycans, less than 0.05% glycosaminoglycans, less than 0.01% glycosaminoglycans, or less than 0.001% glycosaminoglycans. In another specific embodiment, said ECM composition comprises glycosaminoglycans, e.g., 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin, laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin, glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans, and, additionally, comprises no or an undetectable amount of cytokines, growth factors, and/or deoxycholic acid.

In another specific embodiment provided herein is an ECM composition comprising about 30%, about 35%, about 40%, about 45%, about 50%, about 55%, about 60%, about 65%, about 70% or about 72% collagen and about 10%, about 15%, about 18%, about 20%, about 25%, about 30%, or about 35% elastin. In a specific embodiment, said ECM composition is obtained from placental tissue, e.g., human placental tissue. In another specific embodiment, said ECM composition is obtained from the chorion of a placenta, for example, the chorionic plate, e.g., the chorion, for example, the chorionic plate, from a human placenta. In another specific embodiment, said ECM composition comprises a very low amount of fibronectin, e.g., less than 0.1% fibronectin, less than 0.05% fibronectin, less than 0.01% fibronectin, or less than 0.001% fibronectin. In certain embodiments, said ECM composition comprises 0.001 to 0.1% fibronectin, 0.001 to 0.05% fibronectin, 0.001 to 0.01% fibronectin, 0.01 to 0.1% fibronectin, or 0.01 to 0.05% fibronectin. In another specific embodiment, said ECM composition comprises no or an undetectable amount of laminin. In another specific embodiment, said ECM composition comprises a very low amount of laminin, e.g., less than 0.1% laminin, less than 0.05% laminin, less than 0.01% laminin, or less than 0.001% laminin. In another specific embodiment, said ECM composition comprises laminin, e.g., 0.001 to 0.1% laminin, 0.001 to 0.05% laminin, 0.001 to 0.01% laminin, 0.01 to 0.1% laminin, or 0.01 to 0.05% laminin. In another specific embodiment, said ECM composition comprises no or an undetectable amount of glycosaminoglycans. In another specific embodiment, said ECM composition comprises a very low amount of glycosaminoglycans, e.g., less than 0.1% glycosaminoglycans, less than 0.05% glycosaminoglycans, less than 0.01% glycosaminoglycans, or less than 0.001% glycosaminoglycans. In another specific embodiment, said ECM composition comprises glycosaminoglycans, e.g., 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin, laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin, glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans, and, additionally, comprises no or an undetectable amount of cytokines, growth factors, and/or deoxycholic acid.

In another specific embodiment provided herein is an ECM composition comprising about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, 60%, 61%, 62%, 63%, 64%, 65%, 66%, 67%, 68%, 69%, 70%, 71%, or about 72% collagen and about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or 35% elastin. In a specific embodiment, said ECM composition is obtained from placental tissue, e.g., human placental tissue. In another specific embodiment, said ECM composition is obtained from the chorion of a placenta, for example, the chorionic plate, e.g., the chorion, for example, the chorionic plate, from a human placenta. In another specific embodiment, said ECM composition comprises a very low amount of fibronectin, e.g., less than 0.1% fibronectin, less than 0.05% fibronectin, less than 0.01% fibronectin, or less than 0.001% fibronectin. In certain embodiments, said ECM composition comprises 0.001 to 0.1% fibronectin, 0.001 to 0.05% fibronectin, 0.001 to 0.01% fibronectin, 0.01 to 0.1% fibronectin, or 0.01 to 0.05% fibronectin. In another specific embodiment, said ECM composition comprises no or an undetectable amount of laminin. In another specific embodiment, said ECM composition comprises a very low amount of laminin, e.g., less than 0.1% laminin, less than 0.05% laminin, less than 0.01% laminin, or less than 0.001% laminin. In another specific embodiment, said ECM composition comprises laminin, e.g., 0.001 to 0.1% laminin, 0.001 to 0.05% laminin, 0.001 to 0.01% laminin, 0.01 to 0.1% laminin, or 0.01 to 0.05% laminin. In another specific embodiment, said ECM composition comprises no or an undetectable amount of glycosaminoglycans. In another specific embodiment, said ECM composition comprises a very low amount of glycosaminoglycans, e.g., less than 0.1% glycosaminoglycans, less than 0.05% glycosaminoglycans, less than 0.01% glycosaminoglycans, or less than 0.001% glycosaminoglycans. In another specific embodiment, said ECM composition comprises glycosaminoglycans, e.g., 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin, laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin, glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans, and, additionally, comprises no or an undetectable amount of cytokines, growth factors, and/or deoxycholic acid.

The ECM compositions provided herein can be formulated in multiple ways, and the type of formulation can be selected based on, e.g., the intended use of the ECM composition. Exemplary formulations of the ECM compositions provided herein are detailed in Section 4.1.1. In a specific embodiment, the ECM compositions provided herein are formulated as a flowable matrix, e.g., in a form that can be administered using a syringe. In another specific embodiment, the ECM compositions provided herein are formulated as a particulate, e.g., in powder form. In another specific embodiment, the ECM compositions provided herein are formulated as sheets. In certain embodiments, an ECM composition provided herein is formulated to comprise one or more components that are non-ECM components. See Section 4.1.1.1.

In certain embodiments, provided herein are laminates comprising at least one ECM sheet provided herein. In a specific embodiment, provided herein is a laminate comprising two ECM sheets. In another specific embodiment, provided herein is a laminate comprising at least one ECM sheet provided herein and at least one other planar decellularized tissue (e.g., decellularized/dehydrated amniotic membrane, either completely decellularized or decellularized so as to retain a fibroblastic cell layer), or with a planar artificial tissue substitute. In certain embodiments, laminates can be generated by placing ECM sheets, or one or more ECM sheets and another planar decellularized tissues, in contact with each other in the presence of an adhesive, e.g., a glue (e.g., natural glue, e.g., fibronectin, fibrin; or synthetic glue). In certain embodiments, laminates can be generated by heat-drying together two or more ECM sheets, or one or more ECM sheets and one or more planar decellularized tissues.

The ECM compositions provided herein can be seeded with and/or comprise one or more types of cells, i.e., cells can be cultured with and grown upon an ECM composition described herein or dispersed within an ECM composition described herein (e.g., added to an ECM flowable matrix composition). One of skill in the art will appreciate that any cell type known in the art can be seeded with and/or cultured with the ECM compositions provided herein, including both stem cells and non-stem cells. A non-limiting listing of the types of cells that can be seeded on the ECM compositions provided herein is provided in Section 4.1.2.

In another aspect, provided herein are methods of making the ECM compositions described herein. See Section 4.2. In certain embodiments, the methods of making ECM compositions from placenta (e.g., human placenta) that are provided herein comprise the following steps, in order: (i) removing the amnion, chorion, and umbilical cord from a placenta (e.g., from a placenta obtained from a mother immediately after birth, or from a stored placenta); (ii) subjecting the placental tissue to a solution that causes osmotic disruption of cells associated with the placental tissue; (iii) contacting the placenta with a solution comprising a detergent; and (iv) contacting the placenta with a solution comprising a base. In certain embodiments, the methods of making ECM compositions from placenta use the chorion of the placenta (e.g., human placenta), wherein said methods comprise the following steps, in order: (i) obtaining the chorion from a placenta (e.g., from a placenta obtained from a mother immediately after birth, or from a stored placenta); (ii) subjecting the chorion to a solution that causes osmotic disruption of cells associated with the chorion; (iii) contacting the chorion with a solution comprising a detergent; and (iv) contacting the chorion with a solution comprising a base. In certain embodiments, the methods of making ECM compositions from placenta use the chorion of the placenta (e.g., human placenta), wherein said methods comprise the following steps, in order: (i) obtaining the chorion from a placenta (e.g., from a placenta obtained from a mother immediately after birth, or from a stored placenta); (ii) scraping and cleaning the chorion; (iii) subjecting the chorion to a solution that causes osmotic disruption of cells associated with the chorion; (iv) contacting the chorion with a solution comprising a detergent; and (v) grinding and freeze drying. In certain other embodiments, the methods of making ECM compositions from placenta use the chorion of the placenta (e.g., human placenta), wherein said methods comprise the following steps, in order: (i) obtaining the chorion from a placenta (e.g., from a placenta obtained from a mother immediately after birth, or from a stored placenta); (ii) scraping and cleaning the chorion; (iii) subjecting the chorion to a solution that causes osmotic disruption of cells associated with the chorion; (iv) contacting the chorion with a solution comprising with a first, then a second detergent solution, said solutions comprising a detergent and a chelating agent, e.g., EDTA; and (v) freeze drying. The methods of making the ECM compositions described herein use components, e.g., base, detergent, chelating agent, in amounts that result in the generation of ECM compositions having the particular characteristics of those described herein.

The methods of making the ECM compositions described herein use components, e.g., base, detergent, in amounts that result in the generation of ECM compositions having the particular characteristics of those described herein.

In a specific embodiment, provided herein is a method of making an ECM composition, said method comprising (i) removing the amnion, chorion, and umbilical cord from a placenta (e.g., from a placenta obtained from a mother immediately after birth, or from a stored placenta); (ii) placing the remaining placental tissue in a solution that causes osmotic disruption of cells associated with the placental tissue, e.g., sodium chloride (NaCl, e.g., 1 M NaCl) and homogenizing the placental tissue; (iii) contacting the placental tissue with a solution comprising a detergent, e.g., sodium deoxycholate (e.g., 2% sodium deoxycholate); (iv) washing the placental tissue, e.g., with water; (v) contacting the placental tissue with a solution comprising a base, e.g., sodium hydroxide (NaOH, e.g., 1 M NaOH); (vi) adding an acid solution, e.g., hydrochloric acid (HCl), to the solution comprising placental tissue to bring it to or close to a neutral pH (e.g., pH 6.0-8.0); and (vii) separating the placental tissue from the liquid portion of the solution (e.g., by centrifugation) and collecting the placental tissue, thereby making an ECM composition. The ECM composition generated according to the method generally is in the form of a paste (ECM paste), which can be frozen and stored after collection for later use, or which can be used directly after collection to manufacture an ECM formulation described herein, e.g., in the formulation of an ECM sheet, an ECM particulate formulation, or an ECM flowable matrix.

In another specific embodiment, provided herein is a method of making an ECM composition, said method comprising (i) obtaining the chorion from a placenta (e.g., from a placenta obtained from a mother immediately after birth, or from a stored placenta); (ii) placing the chorion tissue in a solution that causes osmotic disruption of cells associated with the chorion tissue, e.g., sodium chloride (NaCl, e.g., 1 M NaCl) and homogenizing the chorion tissue; (iii) contacting the chorion tissue with a solution comprising a detergent, e.g., sodium deoxycholate (e.g., 2% sodium deoxycholate); (iv) washing the chorion tissue, e.g., with water; (v) contacting the chorion tissue with a solution comprising a base, e.g., sodium hydroxide (NaOH, e.g., 1 M NaOH); (vi) adding an acid solution, e.g., hydrochloric acid (HCl), to the solution comprising chorion tissue to bring it to or close to a neutral pH (e.g., pH 6.0-8.0); and (vii) separating the chorion tissue from the liquid portion of the solution (e.g., by centrifugation) and collecting the chorion tissue, thereby making an ECM composition. The ECM composition generated according to the method generally is in the form of a paste (ECM paste), which can be frozen and stored after collection for later use, or which can be used directly after collection to manufacture an ECM formulation described herein, e.g., in the formulation of an ECM sheet, an ECM particulate formulation, or an ECM flowable matrix.

In another specific embodiment, provided herein is a method of making an ECM composition, said method comprising (i) obtaining the chorion, for example, chorionic plate, from a placenta (e.g., from a placenta obtained from a mother immediately after birth, or from a stored placenta); (ii) scraping and cleaning the chorion; (iii) placing the chorion tissue in a solution that causes osmotic disruption of cells associated with the chorion tissue, e.g., sodium chloride (NaCl, e.g., 0.5 M NaCl); (iv) contacting the chorion tissue with a solution comprising a detergent, e.g., deoxycholic acid or sodium deoxycholate (e.g., 2% deoxycholic acid or sodium deoxycholate) and rinsing, e.g., rinsing by water; and (v) grinding and freeze drying. The ECM composition, generally a paste (ECM paste), can be formulated, for example, milled and formulated, into a variety of shapes and forms, e.g., an ECM sheet, an ECM particulate formulation, or an ECM flowable matrix.

In yet another specific embodiment, provided herein is a method of making an ECM composition, said method comprising (i) obtaining the chorion, for example, chorionic plate, from a placenta (e.g., from a placenta obtained from a mother immediately after birth, or from a stored placenta); (ii) scraping and cleaning the chorion; (iii) placing the chorion tissue in a solution that causes osmotic disruption of cells associated with the chorion tissue, e.g., sodium chloride (NaCl, e.g., 1.0 M NaCl); (iv) contacting the chorion tissue with a first detergent solution comprising a detergent, e.g., deoxycholic acid or sodium deoxycholate (e.g., 0.05%-0.2% or 0.3%-0.6% deoxycholic acid or sodium deoxycholate) and EDTA (e.g., 1-5 mM EDTA or 5-10 mM EDTA); (v) contacting the chorion tissue with a second detergent solution comprising a detergent, e.g., deoxycholic acid or sodium deoxycholate (e.g., 0.05%-0.2% or 0.3%-0.6% deoxycholic acid or sodium deoxycholate) and EDTA (e.g., 1-5 mM EDTA or 5-10 mM EDTA), and rinsing, e.g., by water; (vi) freeze drying to yield a decellularized, freeze dried whole chorion which can be formulated, for example, milled and resuspended in solution (e.g., water or phosphate-buffered saline) to form a decellularized ECM paste, and formulated, into a variety of shapes and forms, e.g., an ECM sheet, an ECM particulate formulation, or an ECM flowable matrix.

In a specific embodiment, provided herein is a method of generating an ECM sheet, said method comprising (i) preparing an ECM paste according to the methods described herein; (ii) suspending the ECM paste in, e.g., water, and adding the suspended ECM solution to a suitable substrate for formation of a sheet, e.g., adding the ECM to a mold; (iii) freezing the ECM; (iv) lyophilizing the frozen ECM; (v) removing the lyophilized ECM from the substrate and soaking it in water; and (vi) drying the ECM, e.g., using a vacuum dryer.

In a specific embodiment, provided herein is a method of generating an ECM particulate, said method comprising (i) preparing an ECM paste according to the methods described herein; (ii) suspending the ECM paste in, e.g., water; (iii) freezing the ECM; (iv) lyophilizing the frozen ECM; and (v) milling the lyophilized ECM.

In a specific embodiment, provided herein is a method of generating an ECM flowable matrix, said method comprising (i) preparing an ECM paste according to the methods described herein; (ii) suspending the ECM paste in, e.g., water; (iii) freezing the ECM; (iv) lyophilizing the frozen ECM; and (v) micronizing the lyophilized ECM. Upon resuspension of the micronized ECM in, e.g., saline, an ECM flowable matrix is generated.

In another aspect, provided herein are uses of the ECM compositions provided herein. See Section 4.3. In certain embodiments, the ECM compositions provided herein are used for therapeutic/medical purposes. See Section 4.3.1. In certain embodiments, the ECM compositions provided herein are used for cosmetic purposes. See Section 4.3.2.

In a specific embodiment, the ECM compositions provided herein are used in wound treatment and/or management. See Section 4.3.1. In a specific embodiment, the ECM compositions provided herein are used to fill a wound, that is, as a wound filler. In another specific embodiment, the ECM compositions provided herein are used to dress (i.e., cover) a wound, e.g., a wound caused by a burn.

In another specific embodiment, the ECM compositions provided herein are used in the treatment and/or management of a dental condition, e.g., to repair a dental defect. See Section 4.3.2.

In another specific embodiment, the ECM compositions provided herein are used in the treatment and/or management of oral lesions. See Section 4.3.3.

In another specific embodiment, the ECM compositions provided herein are used to seal, fill, and/or otherwise treat a void within the body of a subject. See Section 4.3.4.

In another specific embodiment, the ECM compositions provided herein are used for tissue bulking in a subject. See Section 4.3.5.

In another specific embodiment, the ECM compositions provided herein are used for treatment of urinary incontinence in a subject. See Section 4.3.6.

In another specific embodiment, the ECM compositions provided herein are used for treatment of vesicoureteral reflux in a subject. See Section 4.3.7.

In another specific embodiment, the ECM compositions provided herein are used for treatment of gastroesophageal reflux disease in a subject. See Section 4.3.8.

In another specific embodiment, the ECM compositions provided herein are used for treatment of a disease, disorder, or other abnormality that affects one or both vocal cords and/or the larynx in a subject. See Section 4.3.9.

In another specific embodiment, the ECM compositions provided herein are used for management or treatment of glottic incompetence in a subject. See Section 4.3.10.

In another specific embodiment, the ECM compositions provided herein are used for bioengineering of tissue or organs. See Section 4.3.11.

In another specific embodiment, the ECM compositions provided herein are for cosmetic purposes, e.g., to augment skin of a subject for a cosmetic purpose (e.g., to make the subject appear more youthful). See Section 4.3.12.

In another aspect, provided herein are kits comprising the ECM compositions provided herein. The kits provided herein typically comprise an ECM composition described herein in a package convenient for distribution to a practitioner of skill in the art. See Section 5.

### 3.1 BRIEF DESCRIPTION OF THE DRAWINGS

FIG. 1A to 1F: Tissue reactivity to ECM Sheet prepared according to the methods described herein as compared to tissue reactivity to porcine urinary bladder matrix following implantation in albino New Zealand White Rabbits. At week 1 postimplantation, the tissue adjacent to the urinary bladder matrix (UBM) sheet showed distinct signs of granulation and inflammatory response (FIG. 1A), while the ECM sheet showed muscle tissue interspersed with slight infiltration of granulocytes (FIG. 1B). At weeks 2 and 4, granulation was still evident adjacent to the UBM sheet (FIGS. 1C and 1E), while tissue adjacent to the ECM sheet showed virtually no granulation and appeared to be normal (FIGS. 1D and 1F).

FIG. 2A to 2F: Tissue reactivity to ECM particulate prepared according to the methods described herein as compared to tissue reactivity to MATRISTEM MICROMATRIX® following implantation in albino New Zealand White Rabbits. At week 1 postimplantation, the ECM particulate showed some granulation indicating inflammation (FIG. 2B), but significantly less than the UBM particulate (FIG. 2A), while at weeks 2 and 4, the ECM particulate showed a significant reduction of granulation (FIGS. 2D and 2F, respectively) as compared to the UBM particulate, which still showed substantial inflammation at weeks 2 and 4 (FIGS. 2C and 2E, respectively).

FIG. 3A to 3F: Tissue reactivity to ECM flowable matrix prepared according to the methods described herein as compared to tissue reactivity to a bovine derived wound matrix product (INTEGRA™ Flowable) following implantation in albino New Zealand White Rabbits. The bovine derived wound matrix product (INTEGRA™ Flowable) showed granulation at week 1 (FIG. 3A), followed by scarring at weeks 2 and 4 (lighter areas in FIGS. 3C and 3E), whereas the ECM flowable matrix showed an inflammatory response substantially only in the first week (FIG. 3B), followed by near-complete healing at weeks 2 and 4 (FIGS. 3D and 3F, respectively).

### 4. DETAILED DESCRIPTION

Provided herein are extracellular matrix (ECM) compositions (see Section 4.1) and methods of making the same (see Section 4.2). Also provided herein are uses of the ECM compositions provided herein (see Section 4.3) and kits comprising the ECM compositions provided herein (see Section 5).

4.1. Extracellular Matrix Compositions

In one aspect, provided herein are extracellular matrix (ECM) compositions prepared using placental tissue, e.g., human placental tissue. The ECM compositions described herein comprise ECM components, e.g., collagen and elastin, in amounts distinct from those in ECM compositions known in the art.

In certain embodiments, the ECM compositions provided herein comprise about 30% to about 60% collagen and about 10% to about 35% elastin. As used herein, the term “about” refers to an amount that is plus or minus 10 percent of a specified number. In addition, such ECM compositions provided herein comprise (i) very low amounts of fibronectin (e.g., less than 0.1% fibronectin), e.g., as measured by ELISA; (ii) no or an undetectable amount of laminin, e.g., as measured by ELISA; and/or no or an undetectable amount of glycosaminoglycans, e.g., as measured by ELISA.

In certain embodiments, the ECM compositions provided herein comprise about 30% to about 72% collagen and about 10% to about 35% elastin. In addition, such ECM compositions can comprise (i) fibronectin (e.g., less than 0.1% fibronectin), e.g., as measured by ELISA; (ii) laminin (e.g., less than 0.1% laminin), e.g., as measured by ELISA; glycosaminoglycans, (e.g., less than 0.1% glycosaminoglycans) e.g., as measured by ELISA; (iii) no or an undetectable amount of cytokines; (iv) no or an undetectable amount of growth factors; and/or (v) no or an undetectable amount of deoxycholic acid.

The ECM compositions provided herein comprise characteristics that make them well-suited for therapeutic/medical use. Specifically, the ECM compositions described herein are sterile, acellular (e.g., ≧99% cell-free) and/or are free of cellular debris (e.g., ≧99% free of cellular debris). In particular embodiments, the ECM compositions provided herein and comprise no cytokines or an undetectable amount of cytokines, as measured by, e.g., ELISA. In certain embodiments, the ECM compositions provided herein further are devoid of reagent residuals, i.e., the final ECM compositions comprise undetectable amounts of reagents used in the manufacture of the compositions. Further, in particular embodiments, the ECM compositions provided herein comprise minimal amounts of nucleic acid (˜41-171 ng/mg of dry product) and endotoxin (<0.25 EU).

Another advantageous characteristic of the ECM compositions provided herein is their ability to absorb water. In certain embodiments, the ECM compositions provided herein absorb between 150%-225% their weight in water.

In a specific embodiment provided herein is an ECM composition comprising 34-53% collagen and 13-29% elastin. In another specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 35-55% collagen and about 10-30% elastin. In another specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In a specific embodiment provided herein is an ECM composition comprising about 35-72% collagen and about 15-25% elastin. In another specific embodiment provided herein is an ECM composition comprising about 40-70% collagen and about 15-25% elastin. In another specific embodiment, said composition comprises 40-70% collagen and 15-22% elastin. In yet another specific embodiment, said composition comprises 43-68% collagen and 18-21% elastin. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin. In another specific embodiment, said ECM composition comprises laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin. In another specific embodiment, said ECM composition comprises glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans. In another specific embodiment, said ECM composition comprises no or an undetectable amount of cytokines, growth factors, and/or deoxycholic acid. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin, laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin, glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans, and, additionally, comprises no or an undetectable amount of cytokines, growth factors, and/or deoxycholic acid. In another specific embodiment, said ECM composition is obtained from placental tissue, e.g., human placental tissue. In another specific embodiment, said ECM composition is obtained from the chorion of a placenta, for example the chorionic plate of a placenta, e.g., the chorion, for example the chorionic plate, from a human placenta.

In another specific embodiment provided herein is an ECM composition comprising about 50-60% collagen and about 10-20% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 45-55% collagen and about 15-25% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 40-50% collagen and about 20-30% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 30-40% collagen and about 25-35% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 34-43% collagen and about 16-24% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 37-42% collagen and about 16-24% elastin. In a specific embodiment, said ECM composition is obtained from placental tissue, e.g., human placental tissue. In another specific embodiment, said ECM composition is obtained from the chorion of a placenta, e.g., the chorion from a human placenta. In another specific embodiment, said ECM composition comprises a very low amount of fibronectin, e.g., less than 0.1% fibronectin, less than 0.01% fibronectin, or less than 0.001% fibronectin. In another specific embodiment, said ECM composition comprises no or an undetectable amount of laminin. In another specific embodiment, said ECM composition comprises no or an undetectable amount of glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans.

In another specific embodiment provided herein is an ECM composition comprising about 30-70%, 30-60%, 30-50%, 30-40%, 30-35%, 34-43%, 35-72%, 35-40%, 37-42%, 40-70%, 40-60%, 40-50%, 40-45%, 40-65%, 43-68%, 45-50%, 50-55%, or 55-60% collagen and about 10-30%, 10-20%, 10-15%, 15-25%, 15-22%, 15-20%, 16-24%, 17-24%, 18-21%, 18-20%, 20-24%, 20-30%, 20-25%, 25-30%, or about 30-35% elastin. In a specific embodiment, said ECM composition is obtained from placental tissue, e.g., human placental tissue. In another specific embodiment, said ECM composition is obtained from the chorion of a placenta, for example the chorionic plate, e.g., the chorion, for example, the chorionic plate, from a human placenta. In another specific embodiment, said ECM composition comprises a very low amount of fibronectin, e.g., less than 0.1% fibronectin, less than 0.05% fibronectin, less than 0.01% fibronectin, or less than 0.001% fibronectin. In certain embodiments, said ECM composition comprises 0.001 to 0.1% fibronectin, 0.001 to 0.05% fibronectin, 0.001 to 0.01% fibronectin, 0.01 to 0.1% fibronectin, or 0.01 to 0.05% fibronectin. In another specific embodiment, said ECM composition comprises no or an undetectable amount of laminin. In another specific embodiment, said ECM composition comprises a very low amount of laminin, e.g., less than 0.1% laminin, less than 0.05% laminin, less than 0.01% laminin, or less than 0.001% laminin. In another specific embodiment, said ECM composition comprises laminin, e.g., 0.001 to 0.1% laminin, 0.001 to 0.05% laminin, 0.001 to 0.01% laminin, 0.01 to 0.1% laminin, or 0.01 to 0.05% laminin. In another specific embodiment, said ECM composition comprises no or an undetectable amount of glycosaminoglycans. In another specific embodiment, said ECM composition comprises a very low amount of glycosaminoglycans, e.g., less than 0.1% glycosaminoglycans, less than 0.05% glycosaminoglycans, less than 0.01% glycosaminoglycans, or less than 0.001% glycosaminoglycans. In another specific embodiment, said ECM composition comprises glycosaminoglycans, e.g., 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin, laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin, glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans, and, additionally, comprises no or an undetectable amount of cytokines, growth factors, and/or deoxycholic acid.

In another specific embodiment provided herein is an ECM composition comprising about 30%, about 35%, about 40%, about 45%, about 50%, about 55%, about 60%, about 65%, about 70% or about 72% collagen and about 10%, about 15%, about 18%, about 20%, about 25%, about 30%, or about 35% elastin. In a specific embodiment, said ECM composition is obtained from placental tissue, e.g., human placental tissue. In another specific embodiment, said ECM composition is obtained from the chorion of a placenta, for example, the chorionic plate, e.g., the chorion, for example, the chorionic plate, from a human placenta. In another specific embodiment, said ECM composition comprises a very low amount of fibronectin, e.g., less than 0.1% fibronectin, less than 0.05% fibronectin, less than 0.01% fibronectin, or less than 0.001% fibronectin. In certain embodiments, said ECM composition comprises 0.001 to 0.1% fibronectin, 0.001 to 0.05% fibronectin, 0.001 to 0.01% fibronectin, 0.01 to 0.1% fibronectin, or 0.01 to 0.05% fibronectin. In another specific embodiment, said ECM composition comprises no or an undetectable amount of laminin. In another specific embodiment, said ECM composition comprises a very low amount of laminin, e.g., less than 0.1% laminin, less than 0.05% laminin, less than 0.01% laminin, or less than 0.001% laminin. In another specific embodiment, said ECM composition comprises laminin, e.g., 0.001 to 0.1% laminin, 0.001 to 0.05% laminin, 0.001 to 0.01% laminin, 0.01 to 0.1% laminin, or 0.01 to 0.05% laminin. In another specific embodiment, said ECM composition comprises no or an undetectable amount of glycosaminoglycans. In another specific embodiment, said ECM composition comprises a very low amount of glycosaminoglycans, e.g., less than 0.1% glycosaminoglycans, less than 0.05% glycosaminoglycans, less than 0.01% glycosaminoglycans, or less than 0.001% glycosaminoglycans. In another specific embodiment, said ECM composition comprises glycosaminoglycans, e.g., 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin, laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin, glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans, and, additionally, comprises no or an undetectable amount of cytokines, growth factors, and/or deoxycholic acid.

In another specific embodiment provided herein is an ECM composition comprising about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, 60%, 61%, 62%, 63%, 64%, 65%, 66%, 67%, 68%, 69%, 70%, 71%, or about 72% collagen and about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or 35% elastin. In a specific embodiment, said ECM composition is obtained from placental tissue, e.g., human placental tissue. In another specific embodiment, said ECM composition is obtained from the chorion of a placenta, for example, the chorionic plate, e.g., the chorion, for example, the chorionic plate, from a human placenta. In another specific embodiment, said ECM composition comprises a very low amount of fibronectin, e.g., less than 0.1% fibronectin, less than 0.05% fibronectin, less than 0.01% fibronectin, or less than 0.001% fibronectin. In certain embodiments, said ECM composition comprises 0.001 to 0.1% fibronectin, 0.001 to 0.05% fibronectin, 0.001 to 0.01% fibronectin, 0.01 to 0.1% fibronectin, or 0.01 to 0.05% fibronectin. In another specific embodiment, said ECM composition comprises no or an undetectable amount of laminin. In another specific embodiment, said ECM composition comprises a very low amount of laminin, e.g., less than 0.1% laminin, less than 0.05% laminin, less than 0.01% laminin, or less than 0.001% laminin. In another specific embodiment, said ECM composition comprises laminin, e.g., 0.001 to 0.1% laminin, 0.001 to 0.05% laminin, 0.001 to 0.01% laminin, 0.01 to 0.1% laminin, or 0.01 to 0.05% laminin. In another specific embodiment, said ECM composition comprises no or an undetectable amount of glycosaminoglycans. In another specific embodiment, said ECM composition comprises a very low amount of glycosaminoglycans, e.g., less than 0.1% glycosaminoglycans, less than 0.05% glycosaminoglycans, less than 0.01% glycosaminoglycans, or less than 0.001% glycosaminoglycans. In another specific embodiment, said ECM composition comprises glycosaminoglycans, e.g., 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans. In another specific embodiment, said ECM composition comprises a very low amount of fibronectin, e.g., less than 0.1% fibronectin, less than 0.01% fibronectin, or less than 0.001% fibronectin; no or an undetectable amount of laminin; and no or an undetectable amount of glycosaminoglycans. In certain embodiments, said ECM composition comprises fibronectin, e.g., less than 0.1% fibronectin, less than 0.05% fibronectin, less than 0.01% fibronectin, less than 0.001% fibronectin, 0.001 to 0.1% fibronectin, 0.001 to 0.05% fibronectin, 0.001 to 0.01% fibronectin, 0.01 to 0.1% fibronectin, or 0.01 to 0.05% fibronectin, laminin, e.g., less than 0.1% laminin, less than 0.05% laminin, less than 0.01% laminin, less than 0.001% laminin, 0.001 to 0.1% laminin, 0.001 to 0.05% laminin, 0.001 to 0.01% laminin, 0.01 to 0.1% laminin, or 0.01 to 0.05% laminin, glycosaminoglycans, e.g., less than 0.1% glycosaminoglycans, less than 0.05% glycosaminoglycans, less than 0.01% glycosaminoglycans, less than 0.001% glycosaminoglycans, 0.001 to 0.1% glycosaminoglycans, 0.001 to 0.05% glycosaminoglycans, 0.001 to 0.01% glycosaminoglycans, 0.01 to 0.1% glycosaminoglycans, or 0.01 to 0.05% glycosaminoglycans, and, additionally, comprises no or an undetectable amount of cytokines, growth factors, and/or deoxycholic acid.

In certain embodiments, the collagen in the ECM compositions described herein comprises or consists of telopeptide collagen. In certain embodiments, the collagen in the ECM compositions described herein comprises or consists of a telopeptide collagen. In certain consists of telopeptide collagen and a telopeptide collagen.

The primary type of collagen in the ECM compositions provided herein is type I collagen. In certain embodiments, the collagen in the ECM compositions provided herein comprises at least 60%, at least 65%, at least 70%, at least 75%, at least 80%, or greater than 80% type I collagen by dry weight. In certain embodiments, the collagen in the ECM compositions provided herein comprises between 50% and 70% type I collagen, between 60% and 80% type I collagen, or between 70% and 90% type I collagen by dry weight. In a specific embodiment, the collagen in the ECM compositions provided herein comprises between 60% and 80% type I collagen.

In certain embodiments, the collagen in the ECM compositions provided herein comprise a mixture of collagen types, e.g., comprises type I collagen as well as type III collagen and/or type IV collagen.

In a specific embodiment, the collagen in the ECM compositions provided herein comprises a substantial amount of type I collagen (e.g., 60%-80% type I collagen) while also comprising type III collagen. For example, in addition to type I collagen, the collagen in the ECM compositions provided herein can comprise between 1% and 5% type III collagen, between 5% and 10% type III collagen, or between 1% and 10% type III collagen by dry weight; or the collagen in the ECM compositions provided herein can comprise about 1%, about 2%, about 3%, about 4%, about 5%, about 6%, about 7%, about 8%, about 9%, or about 10% type III collagen by dry weight.

In another specific embodiment, the collagen in the ECM compositions provided herein comprises a substantial amount of type I collagen (e.g., 60%-80% type I collagen) while also comprising type IV collagen. For example, in addition to type I collagen, the collagen in the ECM compositions provided herein can comprise between 1% and 5% type IV collagen, between 5% and 10% type IV collagen, or between 1% and 10% type IV collagen by dry weight; or the collagen in the ECM compositions provided herein can comprise about 1%, about 2%, about 3%, about 4%, about 5%, about 6%, about 7%, about 8%, about 9%, or about 10% type IV collagen by dry weight.

In another specific embodiment, the collagen in the ECM compositions provided herein comprises a substantial amount of type I collagen (e.g., 60%-80% type I collagen) while also comprising type III collagen and type IV collagen. For example, in addition to type I collagen, the collagen in the ECM compositions provided herein can comprise (i) between 1% and 5% type III collagen, between 5% and 10% type III collagen, or between 1% and 10% type III collagen by dry weight; or the collagen in the ECM compositions provided herein can comprise about 1%, about 2%, about 3%, about 4%, about 5%, about 6%, about 7%, about 8%, about 9%, or about 10% type III collagen by dry weight; and (ii) between 1% and 5% type IV collagen, between 5% and 10% type IV collagen, or between 1% and 10% type IV collagen by dry weight; or the collagen in the ECM compositions provided herein can comprise about 1%, about 2%, about 3%, about 4%, about 5%, about 6%, about 7%, about 8%, about 9%, or about 10% type IV collagen by dry weight.

In certain embodiments, the collagen in the ECM compositions provided herein is cross-linked, e.g., with a cross-linker. Exemplary cross-linkers include glutaraldehyde (see, e.g., U.S. Pat. Nos. 4,852,640, 5,428,022, 5,660,692 and 5,008,116, the contents of which are hereby incorporated by reference in their entirety), 1,4-butanediol diglycidyl ether, and genipin (see, e.g., U.S. Patent Application Publication No. 2003/0049301, the contents of which are hereby incorporated by reference in their entirety). Further exemplary cross-linkers and methods of cross-linking collagen are described in U.S. Pat. Nos. 5,880,242 and 6,117,979 and in Zeeman et al., 2000, J Biomed Mater Res. 51(4):541-8, van Wachem et al., 2000, J Biomed Mater Res. 53(1):18-27, van Wachem et al., 1999, J Biomed Mater Res. 47(2):270-7, Zeeman et al., 1999, J Biomed Mater Res. 46(3):424-33, Zeeman et al., 1999, Biomaterials 20(10):921-31, the contents of which are hereby incorporated by reference in their entireties.

4.1.1 Formulations

The ECM compositions provided herein can be formulated in multiple ways, and the type of formulation can be selected based on, e.g., the intended use of the ECM composition.

In a specific embodiment, the ECM compositions provided herein are formulated as a flowable matrix, e.g., in a form that can be administered using a syringe. Presented herein, therefore, is a syringe comprising an ECM composition as described herein. In certain embodiments, the flowable matrix ECM compositions provided herein can be formulated in water or phosphate buffered saline, e.g., as a solution or suspension, e.g., a mouthwash. When in solution (i.e., as a flowable matrix), an ECM composition provided herein can be present at any concentration useful to one of skill in the art. In certain embodiments, a flowable matrix ECM composition provided herein comprises 200-300 mg/ml, 100-200 mg/ml, 150-250 mg/ml, 0.1-100 mg/ml, 1-100 mg/ml, 1-75 mg/ml, 1-50 mg/ml, 1-40 mg/ml, 10-40 mg/ml or 20-40 mg/ml of ECM. In certain embodiments, a flowable matrix ECM composition provided herein comprises about 5 mg/ml, 10 mg/ml, 15 mg/ml, 20 mg/ml, 25 mg/ml, 30 mg/ml, 35 mg/ml, 40 mg/ml, 45 mg/ml, 50 mg/ml ECM, 75 mg/ml, 100 mg/ml, 125 mg/ml, 150 mg/ml, 175 mg/ml, 200 mg/ml, 225 mg/ml, 250 mg/ml, 275 mg/ml, or 300 mg/ml ECM. In a specific embodiment, provided herein is a flowable matrix ECM composition comprising about 200 mg/ml ECM. In certain embodiments, a flowable matrix ECM composition provided herein is prepared by one of skill in the art using lyophilized, micronized ECM prepared using a method described herein, e.g., said lyophilized, micronized ECM is provided in the form of a kit, accompanied by an appropriate suspension solution, e.g., saline, and one of skill in the art can easily generate a flowable matrix ECM composition by suspending the lyophilized, micronized ECM in the suspension solution.

In another specific embodiment, the ECM compositions provided herein are formulated as a particulate, e.g., in powder form. When present as a particulate, the ECM compositions provided herein can be provided in any container suitable for storage of a particulate, e.g., a vial (e.g., a glass vial). When provided as a particulate, an ECM composition provided herein can be provided in a container at any concentration useful to one of skill in the art. In certain embodiments, a particulate ECM composition provided herein comprises 200-300 mg, 100-200 mg, 150-250 mg, 50-100 mg, 25-50 mg, 10-25 mg, 5-10 mg, or 1-5 mg of ECM. In certain embodiments, a flowable matrix ECM composition provided herein comprises about 5 mg, 10 mg, 15 mg, 20 mg, 25 mg, 30 mg, 35 mg, 40 m, 45 mg, 50 m ECM, 75 mg, 100 mg, 125 mg, 150 mg, 175 mg, 200 mg, 225 mg, 250 mg, 275 mg, or 300 mg ECM. In a specific embodiment, provided herein is a particulate ECM composition comprising about 200 mg ECM. In another specific embodiment, provided herein is a particulate ECM composition comprising about 100 mg ECM

In another specific embodiment, the ECM compositions provided herein are formulated as a sheet, i.e., a planar, solid layer of ECM. When present as a sheet, the ECM compositions provided herein can be shaped into a sheet of any thickness and dimensions suitable for their intended use. In certain embodiments, the ECM sheets provided herein are provided in a standard size, e.g., 5×5 cm or 8×8 cm in size, and can be manipulated (e.g., cut) by one of skill in the art prior to their use, e.g., cut such that they are of suitable size for their intended use, e.g., as a wound dressing. In certain embodiments, the ECM compositions provided herein are formulated as a sheet having a thickness of about 0.1-0.15 mm, 0.15-0.2 mm, 0.1-0.2 mm, 0.2-0.25 mm, 0.25-0.3 mm, or 0.2-0.3 mm. In certain embodiments, the ECM compositions provided herein are formulated as a sheet having a thickness of about 0.1 mm, 0.15 mm, 0.2 mm, 0.25 mm, or 0.3 mm. In certain embodiments, the ECM compositions provided herein are formulated as a 2×2 cm sheet, a 3×3 cm sheet, a 4×4 cm sheet, a 5×5 cm sheet, a 6×6 cm sheet, a 7×7 cm sheet, a 8×8 cm sheet, or a 9×9 cm sheet, wherein said sheet has a thickness of about 0.1-0.15 mm, 0.15-0.2 mm, 0.1-0.2 mm, 0.2-0.25 mm, 0.25-0.3 mm, or 0.2-0.3 mm.

In certain embodiments, provided herein are laminates comprising at least one ECM sheet provided herein. In a specific embodiment, provided herein is a laminate comprising two ECM sheets. In another specific embodiment, provided herein is a laminate comprising at least one ECM sheet provided herein and at least one other planar decellularized tissue (e.g., decellularized/dehydrated amniotic membrane, either completely decellularized or decellularized so as to retain a fibroblastic cell layer), or with a planar artificial tissue substitute. In certain embodiments, laminates can be generated by placing ECM sheets, or one or more ECM sheets and another planar decellularized tissues, in contact with each other in the presence of an adhesive, e.g., a glue (e.g., natural glue, e.g., fibronectin, fibrin; or synthetic glue). In certain embodiments, laminates can be generated by heat-drying together two or more ECM sheets, or one or more ECM sheets and one or more planar decellularized tissues.

4.1.1.1 Non-ECM Components

In certain embodiments, an ECM composition provided herein is formulated to comprise one or more components that are not normally associated with ECM.

In one embodiment, an ECM composition provided herein is combined with a pharmaceutically or cosmetically acceptable carrier, such that the ECM composition is suitable for administration to a subject, e.g., a human subject in need of such administration. Forms of administration include, but are not limited to, injections, solutions, creams, gels, implants, pumps, ointments, emulsions, suspensions, microspheres, particles, microparticles, nanoparticles, liposomes, pastes, patches, tablets, transdermal delivery devices, sprays, aerosols, and other means known to one of skill in the art. Such pharmaceutically or cosmetically acceptable carriers are commonly known to one of ordinary skill in the art. The terms “pharmaceutically or cosmetically acceptable carrier” or “pharmaceutically or cosmetically acceptable vehicle” are used herein to mean, without limitations, any liquid, solid or semi-solid, including, but not limited to, water or saline, a gel, cream, salve, solvent, diluent, fluid ointment base, ointment, paste, implant, liposome, micelle, giant micelle, and the like, which is suitable for use in contact with living animal or human tissue without causing adverse physiological or cosmetic responses, and which does not interact with the other components of the composition, e.g., the ECM, in a deleterious manner.

In one embodiment, an ECM composition provided herein is constituted such that is capable of releasing an active ingredient, e.g., an active ingredient in addition to the ECM composition. For example, an ECM composition provided herein may be impregnated, either during production or afterward, with a biomolecule. Exemplary biomolecules include, but are not limited to, antibiotics (such as clindamycin, minocycline, doxycycline, gentamycin), hormones, growth factors, anti-tumor agents, anti-fungal agents, anti-viral agents, pain medications, anti-histamines, anti-inflammatory agents, anti-infectives, elemental silver, antibiotics, bactericidal enzymes (such as lysozome), wound healing agents (such as cytokines including but not limited to PDGF, TGF; thymosin), hyaluronic acid, wound sealants (such as fibrin with or without thrombin), and cellular attractant and scaffolding reagents. In a specific example, an ECM composition provided herein is impregnated with at least one growth factor, for example, fibroblast growth factor or epithelial growth factor. In certain embodiments, an ECM composition provided herein can be impregnated with small organic molecules, such as specific inhibitors of particular biochemical processes e.g., membrane receptor inhibitors, kinase inhibitors, growth inhibitors, and anticancer drugs.

In certain embodiments, an ECM composition provided herein is combined with a hydrogel. Any hydrogel known to one skilled in the art may be used, e.g., any of the hydrogel compositions disclosed in Graham, 1998, Med. Device Technol. 9(1): 18-22; Peppas et al., 2000, Eur. J. Pharm. Biopharm. 50(1): 27-46; Nguyen et al., 2002, Biomaterials, 23(22): 4307-14; Heninel et al., 2002, Adv. Drug Deliv. Rev 54(1): 13-36; Skelhorne et al., 2002, Med. Device. Technol. 13(9): 19-23; or Schmedlen et al., 2002, Biomaterials 23: 4325-32. In a specific embodiment, the hydrogel is applied onto the ECM composition, i.e., discharged on the surface of the ECM composition. The hydrogel for example, may be sprayed onto the ECM composition, saturated on the surface of the ECM composition, soaked with the ECM composition, bathed with the ECM composition, or coated onto the surface of the ECM composition. The hydrogels useful in the methods and compositions provided herein can be made from any water-interactive, or water soluble polymer known in the art, including but not limited to, polyvinylalcohol (PVA), polyhydroxyethyl methacrylate, polyethylene glycol, polyvinyl pyrrolidone, hyaluronic acid, dextran, and derivatives and analogs thereof.

4.1.2 Cells

The ECM compositions provided herein can comprise, be seeded with, or be cultured with one or more types of cells. For example, cells can be cultured with and grown upon an ECM composition described herein or dispersed on or within an ECM composition described herein (e.g., added to an ECM flowable matrix composition). One of skill in the art will appreciate that any cell type known in the art can be seeded with and/or cultured with the ECM compositions provided herein, including both stem cells and non-stem cells.

In certain embodiments, the ECM compositions provided herein are seeded with and/or comprise stem cells. The stem cells can be any stem cells suitable for a given purpose, and can be totipotent or pluripotent stem cells, or can be progenitor cells. In a specific embodiment, an ECM composition provided herein is seeded with and/or comprises placental stem cells, such as those described in U.S. Pat. Nos. 7,045,148; 7,468,276; 8,057,788 and 8,202,703, the disclosures of which are hereby incorporated by reference in their entireties. In another specific embodiment, an ECM composition provided herein is seeded with and/or comprises embryonic stem cells, embryonic germ cells, mesenchymal stem cells, bone marrow-derived stem cells, hematopoietic progenitor cells (e.g., hematopoietic stem cells from peripheral blood, fetal blood, placental blood, umbilical cord blood, placental perfusate, etc.), somatic stem cells, neural stem cells, hepatic stem cells, pancreatic stem cells, endothelial stem cells, cardiac stem cells, muscle stem cells, adipose stem cells, and the like. In specific embodiments, the stem cells are human stem cells.

In certain embodiments, the ECM compositions provided herein are seeded with and/or comprise one or more types of non-stem cells. As used herein, “non-stem cell” refers to a terminally-differentiated cell. For example, in one embodiment, the ECM compositions provided herein comprise a plurality of fibroblasts. Non-stem cells that can be combined with the ECM compositions provided herein include, without limitation, fibroblasts or fibroblast-like cells, dermal cells, endothelial cells, epithelial cells, muscle cells, cardiac cells, and pancreatic cells. In certain other embodiments, an ECM composition provided herein is seeded with and/or comprises at least two types of non-stem cells.

In certain embodiments, the ECM compositions provided herein are cultured with cells for a time sufficient for a plurality of said cells to attach to the ECM composition. In accordance with such embodiments, the ECM composition can be shaped into a useful configuration, e.g., shaped as a sheet, plug, tube, or other configuration, prior to contacting the ECM composition with the cells.

The ECM compositions can be cultured with at least 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹²; or may be no more than 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹² cells.

4.1.1. Characterization

Biochemical based assays known in the art may be used to confirm the biochemical compositions of the ECM compositions produced using the methods described herein. Protein content can be determined using, e.g., absorbance assays, such as those described in Layne, E, Spectrophotometric and Turbidimetric Methods for Measuring Proteins, Methods in Enzymology 3: 447-455, (1957); Stoscheck, C M, Quantitation of Protein, Methods in Enzymology 182: 50-69, (1990)), Scopes, R K, Analytical Biochemistry 59: 277, (1974); and Stoscheck, C M. Quantitation of Protein, Methods in Enzymology 182: 50-69, (1990)). Alternatively, colorimetric based assays can be used to measure content of particular proteins, included including the modified Lowry assay, biuret assay, Bradford assay, and Bicinchoninic Acid (Smith) assay (see, e.g., Stoscheck, C M, Quantitation of Protein, Methods in Enzymology 182: 50-69 (1990)).

Total collagen content of the ECM compositions provided herein can be determined using, e.g., hydroxyproline or a quantitative dye-based assay kit, e.g., the SIRCOL™ kit manufactured by Biocolor Ltd, UK. Collagen types in the ECM compositions provided herein can be determined using standard methods known in the art, e.g., ELISA assay.

Total elastin content of the ECM compositions provided herein can be determined using, e.g., a quantitative dye-based assay, e.g., the dye-based assay kit (FASTIN) manufactured by Biocolor Ltd, UK.

Total glycosaminoglycan (GAG) content of the ECM compositions provided herein can be determined using, e.g., the quantitative dye-based assay kit (BLYSCAN) manufactured by Biocolor Ltd, UK. GAG content also can be measured by ELISA, using methods known in the art.

Total laminin and fibronectin content of the ECM compositions provided herein can be determined using, for example, an ELISA assay, e.g., a sandwich ELISA assay, e.g., the ELISA assays specific for laminin or fibronectin provided as a kit from Takara Bio Inc., Shiga, Japan.

The ECM compositions described herein are non-immunogenic and biocompatible with tissues of subjects, e.g., human subjects. “Biocompatibility,” as used herein refers to the property of being biologically compatible by not producing a toxic, injurious, or immunological response or rejection in living tissue. Biocompatibility assays can be performed to confirm biocompatibility of the ECM compositions provided herein. Such assays are known to one of skill in the art and include, but are not limited to, cytotoxicity assays (e.g., the ISO MEM Elution test), rabbit eye irritation tests, hemolysis assays, and pyrogencity assays.

The ECM compositions provided herein can be formulated to be sterile, and thus free of microbiological contaminants. Presence of microorganisms can be determined using art-known methods, e.g., direct inoculation of an ECM composition in/on an appropriate bacterial growth medium, e.g., soy casein media or thioglycollate media.

The ECM compositions provided herein can be formulated to comprise little, no, or undetectable levels of endotoxin. Presence of endotoxin in an ECM composition provided herein can be determined using, e.g., the Limulus Amebocyte Lysate (LAL) test (bacterial endotoxin test), an in vitro assay well-known in the art. In a specific embodiment, the ECM compositions provided herein comprise less than 20 endotoxin units (EU) per formulation (e.g., a sheet of ECM provided herein comprises less than 20 EU).

4.2. Methods of Making Extracellular Matrix Compositions

In one aspect, provided herein are methods of generating placental extracellular matrix (ECM) compositions. In certain embodiments, the methods of making ECM compositions from placenta (e.g., human placenta) that are provided herein comprise the following steps, in order: (i) removing the amnion, chorion, and umbilical cord from a placenta (e.g., from a placenta obtained from a mother immediately after birth, or from a stored placenta); (ii) subjecting the placental tissue to a solution that causes osmotic disruption of cells associated with the placental tissue; (iii) contacting the placenta with a solution comprising a detergent; and (iv) contacting the placenta with a solution comprising a base. In certain embodiments, the methods of making ECM compositions from placenta use the chorion of the placenta (e.g., human placenta), wherein said methods comprise the following steps, in order: (i) obtaining the chorion from a placenta (e.g., from a placenta obtained from a mother immediately after birth, or from a stored placenta); (ii) subjecting the chorion to a solution that causes osmotic disruption of cells associated with the chorion; (iii) contacting the chorion with a solution comprising a detergent; and (iv) contacting the chorion with a solution comprising a base. In certain embodiments, the methods of making ECM compositions from placenta use the chorion of the placenta (e.g., human placenta), wherein said methods comprise the following steps, in order: (i) obtaining the chorion from a placenta (e.g., from a placenta obtained from a mother immediately after birth, or from a stored placenta); (ii) scraping and cleaning the chorion; (iii) subjecting the chorion to a solution that causes osmotic disruption of cells associated with the chorion; (iv) contacting the chorion with a solution comprising a detergent; and (v) grinding and freeze drying. In certain other embodiments, the methods of making ECM compositions from placenta use the chorion of the placenta (e.g., human placenta), wherein said methods comprise the following steps, in order: (i) obtaining the chorion from a placenta (e.g., from a placenta obtained from a mother immediately after birth, or from a stored placenta); (ii) scraping and cleaning the chorion; (iii) subjecting the chorion to a solution that causes osmotic disruption of cells associated with the chorion; (iv) contacting the chorion with a solution comprising with a first, then a second detergent solution, said solutions comprising a detergent and a chelating agent, e.g., EDTA; and (v) freeze drying. Importantly, the methods of making the ECM compositions described herein use components, e.g., base, detergent, chelating agent, in amounts that result in the generation of ECM compositions having the particular characteristics of those described herein.

The placentas used in the methods of ECM generation provided herein are generally obtained following a full-term birth, and can be freshly isolated or previously isolated and stored frozen. Generally, when a frozen placenta is used to prepare an ECM composition in accordance with the methods described herein, the placenta is thawed, e.g., at room temperature before use. For example, the placenta can be thawed at ˜22-23° C. for ˜24 hours, or until the placenta is ready for use.

In certain embodiments, prior to preparing an ECM composition from placenta in accordance with the methods provided herein, the placenta is exsanguinated, i.e., drained of the cord blood remaining after birth. In certain embodiments, the placenta is 70% exsanguinated, 80% exsanguinated, 90% exsanguinated, 95% exsanguinated or 99% exsanguinated before use in a method provided herein.

The step of contacting the placental tissue (e.g., placental tissue from which the amnion, chorion, and umbilical cord has been removed; or chorion from placenta) with a solution that causes osmotic disruption of cells associated with the placental tissue being processed in the methods provided herein results in removal of blood and blood components as well as cells and cellular debris that are normally associated with placental tissue. Accordingly, one of skill in the art will understand that any solution capable of causing osmotic disruption of cells can be used in the methods described herein. For example, NaCl, potassium chloride (KCl), ammonium sulfate, a monosaccharide, a disaccharide (e.g., 20% sucrose), a hydrophilic polymer (e.g., polyethylene glycol), glycerol can be used to disrupt cells due to their osmotic potential.

In a specific embodiment, NaCl is used to cause osmotic disruption of cells associated with the placental tissue (e.g., placental tissue from which the amnion, chorion, and umbilical cord has been removed; or chorion from placenta) used in the methods described herein. In a specific embodiment, a solution comprising about 0.25 M, 0.5 M, 0.75 M, 1.0 M, 1.25 M, 1.5 M, 1.75 M, 2 M, 2.25 M or 2.5 M NaCl is used to cause osmotic disruption of cells associated with the placental tissue used in the methods described herein. In a specific embodiment, a solution comprising about 0.25 M to 5 M, about 0.5 M to 4 M, about 0.75 M to 3 M, or about 1.0 M to 2.0 M NaCl is used to cause osmotic disruption of cells associated with the placental tissue used in the methods described herein.

The step of contacting the placental tissue (e.g., placental tissue from which the amnion, chorion, and umbilical cord has been removed; or chorion from placenta) with a solution that causes osmotic disruption can be carried out at any temperature according to the judgment of one of skill in the art. In certain embodiments, the step is carried out at about 0° C. to 30° C., about 5° C. to 25° C., about 5° C. to 20° C., or about 5° C. to 15° C. In certain embodiments, the step is carried out at about 0° C., about 5° C., about 10° C., about 15° C., about 20° C., about 25° C., or about 30° C. In a specific embodiment, the step is carried out at room temperature. In another specific embodiment, the step is carried out at 37° C.±5° C.

The step of contacting the placental tissue (e.g., placental tissue from which the amnion, chorion, and umbilical cord has been removed; or chorion from placenta) with a solution that causes osmotic disruption can be carried out for a suitable time according to the judgment of those of skill in the art. In certain embodiments, the step can be carried out for about 1-24 hours, about 2-20 hours, about 5-15 hours, about 8-12 hours, or about 2-5 hours. In a specific embodiment, the step is carried out for about 18-26 hours at room temperature. In another specific embodiment, the step is carried out for about 18-26 hours at 37° C.±5° C.

The step of contacting placental tissue (e.g., placental tissue from which the amnion, chorion, and umbilical cord has been removed; or chorion from placenta) with a detergent results in removal of cells and cellular debris (e.g., cell membranes) as well as the removal of nucleic acids from the ECM compositions provided herein. Accordingly, the detergent used in the methods described herein can be any detergent known to one of skill in the art to be capable of disrupting cellular or subcellular membranes. In certain embodiments, the detergent is ionic. For instance, in certain embodiments, the detergent is sodium deoxycholate, deoxycholic acid, or sodium dodecylsulfate. In certain embodiments, the detergent is zwitterionic. In certain embodiments, the detergent is nonionic. For instance, in certain embodiments, the detergent can be a TWEEN® detergent, such as TWEEN®-20, or a Triton X detergent, such as Triton X 100. The collagen composition can be contacted with the detergent under conditions judged by one of skill in the art to be suitable for removing unwanted components from the composition.

In a specific embodiment, the detergent used in the methods provided herein is sodium deoxycholate or deoxycholic acid. Said sodium deoxycholate or deoxycholic acid can be used in a method described herein at a suitable concentration for removing cells, cellular debris, and nucleic acid. In particular embodiments of the methods described herein, the sodium deoxycholate or deoxycholic acid can be used, e.g., at a final concentration of about 1.5%, about 2%, or about 2.5%. In a specific embodiment, said sodium deoxycholate or deoxycholic acid is used in a method described herein at a final concentration of about 2%.

In particular other embodiments of the methods described herein, for example, methods that utilize the detergent, e.g., the sodium deoxycholate or deoxycholic acid, together with a chelating agent, for example ethylenediaminetetraacetic acid (EDTA), in a first detergent solution, such sodium deoxycholate or deoxycholic acid can be used, e.g., at a final concentration of about 0.05 to about 0.1%, about 0.05% to about 0.2%, about 0.2 to about 0.3%, about 0.3% to about 0.4%, or about 0.4 to about 0.5%; or at a final concentration of about 0.05%, about 0.06%, about 0.07%, about 0.08%, about 0.09%, about 0.1%, about 0.2%, about 0.3%, about 0.4%, or about 0.5%. In a specific such embodiment, the sodium deoxycholate or deoxycholic acid can be used at a final concentration of about 0.067%. With respect to the chelating agent present in such a first detergent solution, when the chelating agent is EDTA, such EDTA can be used, e.g., at a final concentration of about 1 to about 5 mM, about 2 to about 5 mM, about 2 to about 4 mM, about 3 to about 4 mM, about 3 mM to about 5 mM. about 5 to about 10 mM, about 6 to about 10 mM, about 7 to about 9 mM, or about 8 to about 10 mM; or about 1 mM, about 2 mM, about 3 mM, about 4 mM, about 5 mM, about 6 mM, about 7 mM, about 8 mM, about 9 mM or about 10 mM. In a specific embodiment of the first detergent solution, the solution can comprise sodium deoxycholate or deoxycholic acid at a final concentration of about 0.067% and EDTA at a final concentration of about 4 mM.

In embodiments of the methods described herein, for example, methods that utilize the detergent, e.g., the sodium deoxycholate or deoxycholic acid, together with a chelating agent, for example EDTA, in a second detergent solution, such sodium deoxycholate or deoxycholic acid can be used, e.g., a final concentration of about 0.05 to about 0.1%, about 0.05% to about 0.2%, about 0.2 to about 0.3%, about 0.3% to about 0.4%, or about 0.4 to about 0.5%; or at a final concentration of about 0.05%, about 0.06%, about 0.07%, about 0.08%, about 0.09%, about 0.1%, about 0.2%, about 0.3%, about 0.4%, or about 0.5%. In a specific such embodiment, the sodium deoxycholate or deoxycholic about 0.2% to about 0.6%, acid can be used at a final concentration of about 0.39%. With respect to the chelating agent present in such a second detergent solution, when the chelating agent is EDTA, such EDTA can be used, e.g., at a final concentration of about 1 to about 5 mM, about 2 to about 5 mM, about 2 to about 4 mM, about 3 to about 4 mM, about 3 mM to about 5 mM. about 5 to about 10 mM, about 6 to about 10 mM, about 7 to about 9 mM, or about 8 to about 10 mM; or about 1 mM, about 2 mM, about 3 mM, about 4 mM, about 5 mM, about 6 mM, about 7 mM, about 8 mM, about 9 mM or about 10 mM. In a specific embodiment of the first detergent solution, the solution can comprise sodium deoxycholate or deoxycholic acid at a final concentration of about 0.39% and EDTA at a final concentration of about 8 mM.

The step of contacting placental tissue (e.g., placental tissue from which the amnion, chorion, and umbilical cord has been removed; or chorion from placenta) with a detergent can be carried out at any temperature according to the judgment of one of skill in the art. In certain embodiments, the detergent treatment is carried out at about 0° C. to 30° C., about 5° C. to 25° C., about 5° C. to 20° C., or about 5° C. to 15° C. In certain embodiments, the detergent treatment step is carried out at about 0° C., about 5° C., about 10° C., about 15° C., about 20° C., about 25° C., or about 30° C. In specific embodiment, the detergent treatment step is carried out at room temperature. In another specific embodiment, the detergent treatment step is carried out at 37° C.±5° C.

The detergent treatment (with or without a chelating agent) can be carried out for a suitable time according to the judgment of one of skill in the art. In certain embodiments, the detergent treatment can be carried out for about 1-24 hours, overnight, about 24-48 hours, or about 48-72 hours. In a specific embodiment, the detergent treatment step is carried out for about 72 hours. In another specific embodiment, the detergent treatment step is carried out for about 72 hours at room temperature. In another specific embodiment, the detergent treatment step is carried out for about 72 hours at 37° C.±5° C.

The step of contacting placental tissue (e.g., placental tissue from which the amnion, chorion, and umbilical cord has been removed; or chorion from placenta) with a base results in removal of certain ECM components from the ECM compositions provided herein, e.g., removal of fibronectin and laminin, by denaturing such ECM components. Exemplary bases for the basic treatment include biocompatible bases, volatile bases or bases known to those of skill in the art to be easily and safely removed from the ECM. The base can be any organic or inorganic bases known to those of skill in the art at a concentration of, for example, 0.2 M to 1.0 M. In certain embodiments, the base is ammonium hydroxide, potassium hydroxide or sodium hydroxide, e.g., an ammonium hydroxide solution, potassium hydroxide solution or sodium hydroxide solution.

In a specific embodiment, the base used in the base treatment step of the methods provided herein is sodium hydroxide (NaOH). NaOH can be used in the methods described herein at any concentration suitable for removal of e.g., laminin and fibronectin from the ECM compositions provided herein. For example, NaOH can be used in a method described herein at a concentration of about 0.1 M NaOH, 0.25 M NaOH, 0.5 M NaOH, 1 M NaOH, 1.5 M NaOH, or 2 M NaOH. In a specific embodiment, NaOH is used in a method described herein at a concentration of about 1 M NaOH.

The base treatment step can be carried out at any temperature according to the judgment of one of skill in the art. In certain embodiments, the basic treatment is carried out at about 0° C. to 30° C., about 5° C. to 25° C., about 5° C. to 20° C., or about 5° C. to 15° C. In certain embodiments, the basic treatment is carried out at about 0° C., about 5° C., about 10° C., about 15° C., about 20° C., about 25° C., or about 30° C. In a specific embodiments, the base treatment step is carried out at room temperature. In another specific embodiment, the base treatment step is carried out at 37° C.±5° C.

The base treatment step can be carried out for a suitable time according to the judgment of one of skill in the art. In certain embodiments, the base treatment step can be carried out for about 15 minutes to 30 minutes, 30 minutes to 1 hour, 1 to 2 hours, 2 to 4 hours, 4-8 hours, 8-12 hours, or about 12-24 hours. In a specific embodiment, the base treatment step is carried out for about 30 minutes. In another specific embodiment, the base treatment step is carried out for about 30 minutes at room temperature. In another specific embodiment, the base treatment step is carried out for about 30 minutes at 37° C.±5° C.

In certain embodiments, any or all of the steps of the methods described herein are carried out under sterile conditions. In further embodiments, the ECM compositions prepared according to the methods described herein are further sterilized according to techniques apparent to one of skill in the art and described below.

In a specific embodiment, provided herein is a method of making an ECM composition, said method comprising (i) removing the amnion, chorion, and umbilical cord from a placenta (e.g., from a placenta obtained from a mother immediately after birth, or from a stored placenta); (ii) placing the remaining placental tissue in a solution that causes osmotic disruption of cells associated with the placental tissue, e.g., sodium chloride (NaCl, e.g., 1 M NaCl) and homogenizing the placental tissue; (iii) contacting the placental tissue with a solution comprising a detergent, e.g., sodium deoxycholate (e.g., 2% sodium deoxycholate); (iv) washing the placental tissue, e.g., with water; (v) contacting the placental tissue with a solution comprising a base, e.g., sodium hydroxide (NaOH, e.g., 1 M NaOH); (vi) adding an acid solution, e.g., hydrochloric acid (HCl), to the solution comprising placental tissue to bring it to or close to a neutral pH (e.g., pH 6.0-8.0); and (vii) separating the placental tissue from the liquid portion of the solution (e.g., by centrifugation) and collecting the placental tissue, thereby making an ECM composition. The ECM composition generated according to the method generally is in the form of a paste (ECM paste), which can be frozen and stored after collection for later use, or which can be used directly after collection to manufacture an ECM formulation described herein, e.g., in the formulation of an ECM sheet, an ECM particulate formulation, or an ECM flowable matrix.

In another specific embodiment, provided herein is a method of making an ECM composition, said method comprising (i) removing the amnion, chorion, and umbilical cord from a placenta (e.g., from a placenta obtained from a mother immediately after birth, or from a stored placenta); (ii) cutting the placenta into strips, e.g., 2×2 centimeter strips; (iii) placing the placental tissue in a 1.5 liter 1 M NaCl solution and homogenizing the placental tissue (e.g., using an Omni Mixer Homogenizer (Omni International, Kennesaw, Ga.)), (iv) placing the homogenized placental tissue in a processing receptacle (e.g., a bag) and adding 1 M NaCL to a volume of 9.2 liters; (v) washing the homogenized placental tissue three times with 1 M NaCl, wherein said washing comprises (a) agitating the processing receptacle on a shaker for 10 minutes, (b) allowing placental tissue to settle in the processing receptacle for 10 minutes, and (c) removing 6.2 liters of the supernatant by gravity drainage; (vi) allowing the washed placental tissue to mix in the remaining 1 M NaCl solution on a shaker for ˜18-26 hours at room temperature (vii) washing the placental tissue four times with water, as described above; (viii) allowing the washed placental tissue to mix in water on a shaker for ˜18-26 hours at room temperature; (ix) after the overnight mixing in water, washing a final time with water, as described above, then adding 6.2 L of 3% sodium deoxycholate to the mixture, for a concentration of 2% sodium deoxycholate; (x) allowing the placental tissue to mix in the 2% sodium deoxycholate solution on a shaker for ˜72 hours at room temperature; (xi) washing the placental tissue in water five times, in the manner described above; (xii) after the final addition of water, adjusting the pH of the solution to about 10-12 by addition of 1 M NaOH, resulting in a basic solution; (xiii) allowing the placental tissue to mix in the basic solution on a shaker for ˜30 minutes at room temperature; (xiv) adjusting the pH of the solution to about 7.0-7.5 using 0.1 N HCl; and (xv) removing the supernatant from the processing receptacle (e.g., by centrifugation) and collecting the placental tissue, thereby making an ECM composition.

In a specific embodiment, provided herein is a method of making an ECM composition, said method comprising (i) obtaining the chorion from a placenta (e.g., from a placenta obtained from a mother immediately after birth, or from a stored placenta); (ii) placing the placental chorion in a solution that causes osmotic disruption of cells associated with the placental chorion, e.g., sodium chloride (NaCl, e.g., 1 M NaCl) and homogenizing the placental chorion; (iii) contacting the placental chorion tissue with a solution comprising a detergent, e.g., sodium deoxycholate (e.g., 2% sodium deoxycholate); (iv) washing the placental chorion tissue, e.g., with water; (v) contacting the placental chorion tissue with a solution comprising a base, e.g., sodium hydroxide (NaOH, e.g., 1 M NaOH); (vi) adding an acid solution, e.g., hydrochloric acid (HCl), to the solution comprising placental chorion tissue to bring it to or close to a neutral pH (e.g., pH 6.0-8.0); and (vii) separating the placental chorion tissue from the liquid portion of the solution (e.g., by centrifugation) and collecting the placental chorion tissue, thereby making an ECM composition. The ECM composition generated according to the method generally is in the form of a paste (ECM paste), which can be frozen and stored after collection for later use, or which can be used directly after collection to manufacture an ECM formulation described herein, e.g., in the formulation of an ECM sheet, an ECM particulate formulation, or an ECM flowable matrix. In certain embodiments, the various steps of the method (e.g., osmotic disruption, detergent treatment, washing, base treatment) are carried out at room temperature. In certain embodiments, the various steps of the method (e.g., osmotic disruption, detergent treatment, washing, base treatment) are carried out at 37° C.±5° C. In certain embodiments, the washing step is carried out using a volume of ½ to 2 liters of fluid for 1-3 times.

In another specific embodiment, provided herein is a method of making an ECM composition, said method comprising (i) obtaining the chorion from a placenta (e.g., from a placenta obtained from a mother immediately after birth, or from a stored placenta); (ii) cutting the chorion into strips, e.g., 2×2 centimeter strips; (iii) placing the placental chorion tissue in a 1.5 liter 1 M NaCl solution and homogenizing the placental chorion tissue (e.g., using an Omni Mixer Homogenizer (Omni International, Kennesaw, Ga.)), (iv) placing the homogenized placental chorion tissue in a processing receptacle (e.g., a bag) and adding 1 M NaCL to a volume of 9.2 liters; (v) washing the homogenized placental chorion tissue three times with 1 M NaCl, wherein said washing comprises (a) agitating the processing receptacle on a shaker for 10 minutes, (b) allowing placental chorion tissue to settle in the processing receptacle for 10 minutes, and (c) removing 6.2 liters of the supernatant by gravity drainage; (vi) allowing the washed placental chorion tissue to mix in the remaining 1 M NaCl solution on a shaker for ˜18-26 hours at room temperature (vii) washing the placental chorion tissue four times with water, as described above; (viii) allowing the washed placental chorion tissue to mix in water on a shaker for ˜18-26 hours at room temperature; (ix) after the overnight mixing in water, washing a final time with water, as described above, then adding 6.2 L of 3% sodium deoxycholate to the mixture, for a concentration of 2% sodium deoxycholate; (x) allowing the placental chorion tissue to mix in the 2% sodium deoxycholate solution on a shaker for ˜72 hours at room temperature; (xi) washing the placental chorion tissue in water five times, in the manner described above; (xii) after the final addition of water, adjusting the pH of the solution to about 10-12 by addition of 1 M NaOH, resulting in a basic solution; (xiii) allowing the placental chorion tissue to mix in the basic solution on a shaker for ˜30 minutes at room temperature; (xiv) adjusting the pH of the solution to about 7.0-7.5 using 0.1 N HCl; and (xv) removing the supernatant from the processing receptacle (e.g., by centrifugation) and collecting the placental chorion tissue, thereby making an ECM composition. In certain embodiments, the washing step is carried out using a volume of ½ to 2 liters of fluid.

In another specific embodiment, provided herein is a method of making an ECM composition, said method comprising (i) obtaining the chorion, for example, chorionic plate, from a placenta (e.g., from a placenta obtained from a mother immediately after birth, or from a stored placenta); (ii) scraping and cleaning the chorion; (iii) placing the chorion tissue in a solution that causes osmotic disruption of cells associated with the chorion tissue, e.g., sodium chloride (NaCl, e.g., 0.5 M NaCl); (iv) contacting the chorion tissue with a solution comprising a detergent, e.g., deoxycholic acid or sodium deoxycholate (e.g., 2% deoxycholic acid or sodium deoxycholate) and rinsing, e.g., rinsing by water, and (v) grinding and freeze drying. The ECM composition, generally a paste (ECM paste) can be formulated, for example, milled and formulated, into a variety of shapes and forms, e.g., an ECM sheet, an ECM particulate formulation, or an ECM flowable matrix. In certain embodiments, the various steps of the method (e.g., osmotic disruption, detergent treatment, rinsing) are carried out at room temperature. In certain embodiments, the various steps of the method (e.g., osmotic disruption, detergent treatment, rinsing) are carried out at 37° C.±5° C. In certain embodiments, the rinsing step is carried out using a volume of ½ to 2 liters of fluid for 1-3 times.

In a particular embodiment, provided herein is a method of making an ECM composition, said method comprising, in order: (i) obtaining a human placenta from a mother immediately after a full-term birth, or obtaining a previously isolated frozen human placenta that has been allowed to thaw, for example, allowed to thaw at room temperature for approximately 24 hours; (ii) washing the placenta is washed in 0.5 M NaCl; (iii) removing the amnion, umbilical cord and decidua parietalis from the placenta, and retaining the chorionic plate of the placenta; (iv) scraping and cleaning the chorion; (v) rinsing the chorion in 0.5 M NaCl and water; (vi) rinsing the chorion overnight in 2% deoxycholic acid, followed by multiple water rinses, e.g., 3, 4, 5, 6, 7 or more water rinses; and (vii) grinding and freeze drying the treated chorion. The ECM composition, generally a paste (ECM paste) can be formulated, for example, milled and formulated, into a variety of shapes and forms, e.g., an ECM sheet, an ECM particulate formulation, or an ECM flowable matrix. In certain embodiments, the various steps of the method (e.g., NaCl treatment, deoxycholic acid treatment, rinsing) are carried out at room temperature. In certain embodiments, the various steps of the method (e.g., NaCl treatment, deoxycholic acid treatment, rinsing) are carried out at 37° C.±5° C. In certain embodiments, the rinsing step is carried out using a volume of ½ to 2 liters of fluid for 1-3 times.

In yet another specific embodiment, provided herein is a method of making an ECM composition, said method comprising (i) obtaining the chorion, for example, chorionic plate, from a placenta (e.g., from a placenta obtained from a mother immediately after birth, or from a stored placenta); (ii) scraping and cleaning the chorion; (iii) placing the chorion tissue in a solution that causes osmotic disruption of cells associated with the chorion tissue, e.g., sodium chloride (NaCl, e.g., 1.0 M NaCl); (iv) contacting the chorion tissue with a first detergent solution comprising a detergent, e.g., deoxycholic acid or sodium deoxycholate (e.g., 0.05%-0.2% or 0.3%-0.6% deoxycholic acid or sodium deoxycholate) and EDTA (e.g., 1-5 mM EDTA or 5-10 mM EDTA); (v) contacting the chorion tissue with a second detergent solution comprising a detergent, e.g., deoxycholic acid or sodium deoxycholate (e.g., 0.05%-0.2% or 0.3%-0.6% deoxycholic acid or sodium deoxycholate) and EDTA (e.g., 1-5 mM EDTA or 5-10 mM EDTA), and rinsing, e.g., by water; and (vi) freeze drying to yield a decellularized, freeze dried whole chorion which can be formulated, for example, milled into and resuspended in solution (e.g., water or phosphate-buffered saline) to form a decellularized ECM paste, and formulated, into a variety of shapes and forms, e.g., an ECM sheet, an ECM particulate formulation, or an ECM flowable matrix. In certain embodiments, the various steps of the method (e.g., osmotic disruption, detergent/EDTA treatment, rinsing) are carried out at room temperature. In certain embodiments, the various steps of the method (e.g., osmotic disruption, detergent/EDTA treatment, rinsing) are carried out at 37° C.±5° C. In certain embodiments, the rinsing step is carried out using a volume of ½ to 2 liters of fluid for 1-3 times.

In a particular embodiment, provided herein is a method of making an ECM composition, said method comprising, in order: (i) obtaining a human placenta from a mother immediately after a full-term birth, or obtaining a previously isolated frozen human placenta that has been allowed to thaw, for example, allowed to thaw at room temperature for approximately 24 hours; (ii) removing the amnion, umbilical cord and decidua parietalis from the placenta, and retaining the chorionic plate of the placenta; (iii) scraping and cleaning the chorion; (iv) rinsing the chorion in 1.0 M NaCl and water; (v) rinsing the chorion overnight in a first detergent solution comprising 0.067% deoxycholic acid and 4 mM EDTA, followed by multiple water rinses, e.g., 3, 4, 5, 6, 7 or more water rinses; (vi) rinsing the chorion overnight in a second detergent solution comprising 0.39% deoxycholic acid and 8 mM EDTA, followed by multiple water rinses, e.g., 3, 4, 5, 6, 7 or more water rinses; and (vii) freeze drying the treated chorion. The resulting composition is a decellularized ECM paste suitable for further formulation, e.g., milling and formulation. In certain embodiments, the various steps of the method (e.g., NaCl treatment, deoxycholic acid/EDTA treatment, rinsing) are carried out at room temperature. In certain embodiments, the various steps of the method (e.g., NaCl treatment, deoxycholic acid/EDTA treatment, rinsing) are carried out at 37° C.±5° C. In certain embodiments, the rinsing step is carried out using a volume of ½ to 2 liters of fluid for 1-3 times.

In a specific embodiment, provided herein is a method of generating an ECM sheet, said method comprising (i) preparing an ECM paste according to the methods described herein; (ii) suspending the ECM paste in, e.g., water, and adding the suspended ECM solution to a suitable substrate for formation of a sheet, e.g., adding the ECM to a mold; (iii) freezing the ECM (iv) lyophilizing the frozen ECM; (v) removing the lyophilized ECM from the substrate and soaking it in water; and (vi) drying the ECM, e.g., using a vacuum dryer. After rehydration, and prior to drying, the ECM sheets provided herein can be formed into any useful shape, e.g., a block, a tube, or another shape.

In a specific embodiment, provided herein is a method of generating an ECM particulate, said method comprising (i) preparing an ECM paste according to the methods described herein; (ii) suspending the ECM paste in, e.g., water; (iii) freezing the ECM (iv) lyophilizing the frozen ECM; and (v) milling the lyophilized ECM.

In a specific embodiment, provided herein is a method of generating an ECM flowable matrix, said method comprising (i) preparing an ECM paste according to the methods described herein; (ii) suspending the ECM paste in, e.g., water; (iii) freezing the ECM (iv) lyophilizing the frozen ECM; and (v) micronizing the lyophilized ECM. Upon resuspension of the micronized ECM in, e.g., saline, an ECM flowable matrix is generated.

Lyophilization of the ECM compositions produced according to the methods described herein can be accomplished by any means known in the art, and generally proceeds until the ECM composition is substantially dry, e.g., less than about 30%, 25%, 20%, 25%, 20%, 5%, 4%, 3%, 2% or 1% water by weight.

4.2.1. Optional Further Treatment

In certain embodiments, the methods provided herein incorporate an additional step, e.g., a step that results in treatment of the ECM composition being prepared with another agent other than a solution capable of causing osmotic disruption of cells, a detergent, or a base.

In a specific embodiment, the methods provided herein comprise treatment of an ECM composition being prepared according to the methods described herein with BENZONASE®. BENZONASE® is a genetically engineered endonuclease derived from Serratia marcescens that attacks and degrades all forms of DNA and RNA. Accordingly, BENZONASE® can be used in accordance with the methods described herein to ensure that the resulting ECM composition are free of (or substantially free of) nucleic acid. After treatment with BENZONASE®, the ECM composition treated can be brought to a high pH, then a low pH, conditions suitable for inactivation of BENZONASE®.

In a specific embodiment, the methods provided herein comprise treatment of an ECM composition being prepared according to the methods described herein with ethylenediaminetetraacetic acid (EDTA). EDTA is a metal chelator well-known to one of skill in the art, and can be used in accordance with the methods provided herein to remove divalent metal ions from the ECM compositions provided herein. EDTA can be washed out of the ECM compositions using methods known to one of skill in the art.

In certain embodiments, the collagen in the ECM compositions provided herein can be cross-linked. The cross-linking can be with any cross-linker known to one of skill in the art, for instance, the cross-linkers described above. In certain embodiments, the cross-linker is glutaraldehyde, and the cross-linking can be carried out according to methods of glutaraldehyde cross-linking of collagen known to one of skill in the art. In other embodiments, the cross-linker is 1,4-butanediol diglycidyl ether or genipin.

4.2.2. Storage

In certain embodiments, the ECM compositions provided herein are stored at room temperature (e.g., ˜22-25° C.). In certain embodiments, the ECM compositions provided herein are stored cold, e.g., refrigerated at a temperature of about 0° C., about 4° C., or about 8° C. In some embodiments, the ECM is not refrigerated. In certain embodiments, the ECM compositions provided herein are stored frozen, i.e., at a temperature below 0° C., e.g., at −10° C., −15° C., −20° C., −25° C., −30° C., −35° C., −40° C., −45° C., −50° C., −55° C., −60° C., −65° C., −70° C., −75° C., −80° C., or −85° C., or colder. In certain embodiments, said freezing and storage of the ECM compositions provided herein takes place at a temperature between 0° C. to −10° C., −10° C. to −20° C., −20° C. to −30° C., −30° C. to −40° C., −40° C. to −50° C., −50° C. to −60° C., −60° C. to −70° C., or −70° C., to −80° C.

In certain embodiments, the ECM compositions provided herein are stored under sterile and non-oxidizing conditions. In certain embodiments, the ECM compositions provided herein are stored at any of the above-specified temperatures for 12 months or more.

4.2.3. Sterilization

In certain embodiments, the ECM compositions provided herein are sterilized according to techniques known to those of skill in the art for sterilizing such compositions. In a specific embodiment, the ECM compositions provided herein are sterilized by radiation, e.g., gamma irradiation.

In a specific embodiment, sterilization of the ECM compositions provided herein is carried out by electron beam irradiation using methods known to one skilled in the art, see, e.g., Gorham, D. Byrom (ed.), 1991, Biomaterials, Stockton Press, New York, 55-122. Any dose of radiation sufficient to kill at least 99.9% of bacteria or other potentially contaminating organisms is within the scope of the methods provided herein. In a particular embodiment, a dose of at least 18-25 kGy is used to achieve terminal sterilization of an ECM composition provided herein.

In certain embodiments, the ECM compositions provided herein are filtered through a filter that allows passage of endotoxins and retains the ECM composition. Any filter of a size, for example 30 kDa, known to one of skill in the art for filtration of endotoxins can be used. In certain embodiments, the filter is between 5 kDa and 100 kDa, e.g., the filter is about 5 kDa, about 10 kDa, about 15 kDa, about 20 kDa, about 30 kDa, about 40 kDa, about 50 kDa, about 60 kDa, about 70 kDa, about 80 kDa, about 90 kDa or about 100 kDa. The filter can be of any material known to those of skill in the art to be compatible with the ECM compositions provided herein, such as cellulose or polyethersulfone. The filtration can be repeated as many times as desired by one of skill in the art. Endotoxin can be detected according to standard techniques to monitor clearance.

In certain embodiments, the ECM compositions provided herein are filtered to generate ECM compositions free of, or reduced in, viral particles. Any filter known to one of skill in the art to be useful for clearing viruses can be used. For instance, a 1000 kDa filter can be used for clearance, or reduction, of parvovirus, hepatitis A virus and HIV. A 750 kDa filter can be used for clearance, or reduction, of parvovirus and hepatitis A virus. A 500 kDa filter can be used for clearance, or reduction, of parvovirus. The filter can be of any material known to those of skill in the art to be compatible with the ECM compositions provided herein, such as cellulose or polyethersulfone. The filtration can be repeated as many times as desired by one of skill in the art. Presence of virus can be detected according to standard techniques to monitor clearance.

4.3. Uses

The ECM compositions provided herein (see Section 4.1) can be used in numerous ways and for many purposes, including, but not limited to, use of the ECM compositions in the manufacture of engineered tissue and organs, including structures such as patches or plugs of tissues or matrix material, prosthetics, and other implants; use of the ECM compositions as tissue scaffolding; use of the ECM compositions in the repair of or dressing of wounds; use of the ECM compositions as hemostatic devices; use of the ECM compositions as devices for use in tissue repair and support, such as sutures, surgical and orthopedic screws, surgical and orthopedic plates, natural coatings or components for synthetic implants, cosmetic implants and supports; use of the ECM compositions in the repair of or as structural support for organs or tissues; use of the ECM compositions for substance delivery; use of the ECM compositions as bioengineering platforms; use of the ECM compositions as platforms for testing the effect of substances upon cells; and use of the ECM compositions in cell culture. Further, the ECM compositions can be used for cosmetic purposes.

In certain embodiments, use of the ECM compositions provided herein requires administration of an ECM composition provided herein to a subject. As used herein, the term “subject” refers to animals, such as mammals, including, but not limited to, primates (e.g., humans), cows, sheep, goats, horses, dogs, cats, rabbits, rats, mice and the like. In a specific embodiment, the subject is a human.

Methods of in vivo administration of the ECM compositions provided herein to a subject include, but are not limited to, oral administration (e.g. buccal or sublingual administration), topical application, aerosol application, transdermal administration, intradermal administration, subdermal administration, intramuscular administration, and surgical administration.

In certain embodiments, as part of a use provided herein, the ECM compositions provided herein can be applied in the form of creams, gels, solutions, suspensions, liposomes, particles, or other means known to one of skill in the art of formulation and delivery of therapeutic and cosmetic compounds. Some examples of appropriate formulations for subcutaneous administration include but are not limited to implants, depot, needles, capsules, and osmotic pumps. Some examples of appropriate formulations for oral administration include but are not limited to: pastes, patches, sheets, liquids, syrups, suspensions, aerosols and mists. Some examples of appropriate formulations for transdermal administration include but are not limited to creams, pastes, patches, sprays, and gels. Some examples of appropriate delivery mechanisms for subcutaneous administration include but are not limited to implants, depots, needles, capsules, and osmotic pumps.

The ECM compositions provided herein can be administered to a subject in any form and/or concentration that will produce desired physiological or pharmacological results. Form and concentration of the ECM composition will depend upon therapeutic endpoint desired, the desired effective concentration at the site of action or in a body fluid, and the type of administration. Information regarding administration of substances to subjects is known to persons of ordinary skill in the art and may be found in references such as L. S. Goodman and A. Gilman, eds, The Pharmacological Basis of Therapeutics, Macmillan Publishing, New York, and Katzung, Basic & Clinical Pharmacology, Appleton & Lang, Norwalk, Conn., (6th Ed. 1995). A clinician skilled in the art of the desired therapy may chose specific form and concentrations, and frequency of administration, as required by the circumstances and the substances to be administered.

4.3.1. Use in Treatment of Wounds

In certain embodiments, the ECM compositions provided herein are used in the treatment of wounds.

In one embodiments, the ECM compositions provided herein are used to treat a wound by placing the ECM composition directly over the skin of the subject at the site of the wound, so that the wound is covered. In another embodiment, the ECM compositions provided herein are used to treat a wound by using the ECM composition as an implant, e.g., as a subcutaneous implant. One of skill in the art will recognize that certain formulations of the ECM composition are suitable for such uses. For example, an ECM composition formulated as a sheet can be used to treat a wound, by placing the ECM sheet over the wound on the skin of a subject.

In certain embodiments, when used in the treatment of a wound, an ECM composition provided herein can be formulated to comprise one or more pharmacologically active agents including, but not limited to, platelet-derived growth factor, insulin-like growth factor, epidermal growth factor, transforming growth factor beta, angiogenesis factor, antibiotics, antifungal agents, spermicidal agents, hormones, enzymes, and enzyme inhibitors.

Wounds that can be treated with the ECM compositions provided herein include, but are not limited to, epidermal wounds, skin wounds, chronic wounds, acute wounds, external wounds, internal wounds (e.g., an ECM composition may be wrapped around an anastosmosis site during surgery to prevent leakage of blood from suture lines, and to prevent the body from forming adhesions to the suture material), congenital wounds (e.g., dystrophic epidermolysis bullosa), pressure ulcers (e.g., decubitus ulcers), partial and full-thickness wounds, venous ulcers, diabetic ulcers, chronic vascular ulcers, tunneled/undermined wounds, surgical wounds (e.g., donor sites/grafts, post-Moh's surgery, post-laser surgery, podiatric, wound dehiscence), trauma wounds (e.g., abrasions, lacerations, second-degree burns, and skin tears), and draining wounds.

In a specific embodiment, the ECM compositions provided herein are used in the treatment of burns and/or conditions associated with burns, including, but not limited to, first-degree burns, second-degree burns (partial thickness burns), third degree burns (full thickness burns), infections of burn wounds, infection of excised and unexcised burn wounds, loss of epithelium from a previously grafted or healed burn wound, and burn wound impetigo.

4.3.2. Dental

In certain embodiments, the ECM compositions provided herein are used in the treatment of dental diseases and disorders.

In one embodiment, the ECM compositions provided herein are used in periodontal surgery, guided tissue regeneration for regeneration of periodontal tissue, guided bone regeneration, and/or root coverage. Such methods encompass the use of the ECM compositions to promote regeneration of periodontal intrabony defects, including but not limited to matched bilateral periodontal defects, interdental intrabony defects, deep 3-wall intrabony defects, 2-wall intrabony defects, and intrabony defects 2 and 3.

In another embodiment, the ECM compositions provided herein are used in the treatment of class II furcation defects including but not limited to bilateral defects, paired buccal Class II mandibular molar furcation defects, and bilateral mandibular furcation defect.

In another embodiment, the ECM compositions provided herein are used in the treatment of periodontal disease including but not limited to, periodontitis and gingivitis. In one embodiment, an ECM composition provided herein can be used to treat a subject with a periodontal disease by, e.g., inserting the ECM composition, which can be impregnated with an antibiotic such as chlorhexidine gluconate, into one or more periodontal pockets in the subject, e.g., greater than or equal to 5 mm.

4.3.3 Oral Lesions

In certain embodiments, the ECM compositions provided herein are used in the treatment of oral lesions, wherein said lesions are not caused by a dental procedure or by oral surgery. In certain embodiments, provided herein is a method of treating a subject who has an oral lesion comprising administering to the subject, e.g., administering to the oral lesion, a therapeutically-effective amount of an ECM composition provided herein. In this context, “therapeutically effective amount” means an amount of an ECM composition that acts to reduce or eliminate at least one symptom or aspect of the oral lesion. For example, the ECM composition can be administered in order to repair the lesion, or can be administered as a palliative, e.g., to reduce pain or inflammation caused by or associated with the oral lesion.

In certain embodiments, an ECM composition provided herein is administered directly into an oral lesion as a means of treatment. In other embodiments, an ECM composition provided herein is administered adjacent to or at the periphery of at least a part of the oral lesion. Such administration can be, for example, by placement of a sheet of the ECM composition over at least a portion, or the whole of, the oral lesion. In certain embodiments, the ECM composition is administered to the oral lesion as a paste. In certain embodiments, the ECM composition is administered to the oral lesion in the form of a spray or aerosol. In certain embodiments, the ECM composition is administered to the oral lesion in the form of a solution, e.g., in a mouthwash.

Oral lesions treated in accordance with the methods provided herein can be caused by any condition or treatment known in the art to cause oral lesions. In one embodiment, an oral lesion treated using an ECM composition provided herein is caused by or associated with desquamation, e.g., a desquamating oral disorder. In another embodiment, an oral lesion treated using an ECM composition provided herein is an aphthous ulcer, e.g., an aphthous ulcer caused by, or part of, Behçet's disease. In another embodiment, an oral lesion treated using an ECM composition provided herein is caused by, or is associated with, osteonecrosis of the jaw of a subject.

In another embodiment, an oral lesion treated using an ECM composition provided herein is caused by or is associated with, graft-versus-host disease. In another embodiment, an oral lesion treated using an ECM composition provided herein is caused by or associated with use of melphalan by the subject having the oral lesion.

In another embodiment, an oral lesion treated using an ECM composition provided herein is caused by or associated with chemotherapy, e.g., chemotherapy that has been administered to the subject to treat a tumor, blood cancer, or other type of cancer. In a specific embodiment, the oral lesion is caused by post-chemotherapy oral mucositis or chemotherapy-induced oral mucositis. In another specific embodiment, the oral lesion is, or is diagnosed as, aphthous stomatitis, e.g., idiopathic aphthous stomatitis. In another specific embodiment, the oral lesion is caused by or associated with use of an mTOR (mammalian target of rapamycin) inhibitor by the subject having the oral lesion. In another specific embodiment, the oral lesion is caused by or associated with use of 5-fluorouracil by the subject having the oral lesion. In specific embodiments, in which the oral lesion is caused by or associated with chemotherapy, e.g., is caused by or associated with use of a chemotherapeutic agent, the chemotherapeutic agent is, e.g., an alkylating agent (e.g., busulfan, cisplatin, carboplatin, cyclophosphamide, dacarbazine, ifosfamide, mechlorethamine or melphalan); an anti-metabolite (e.g., 5-fluorouracil, methotrexate, gemcitabine, cytarabine, or fludarabine); antibiotics having an antitumor effect (e.g., bleomycin, dactinomycin, daunorubicin, doxorubicin, or idarubicin); or mitotic inhibitors (e.g., paclitaxel, docetaxel, etoposide, vinblastine, vincristine or vinorelbine). In another specific embodiment, development of said oral lesion in said subject, wherein said subject is receiving or has received a course of therapy, e.g., chemotherapy, has caused, or is expected to cause, a premature termination of said course of therapy. In this context, “premature termination” means termination of the course of therapy prior to what has been prescribed for said subject, partially or wholly as a result of said oral lesion.

In another embodiment, an oral lesion treated using an ECM composition provided herein is caused by or associated with administration of an antibody to a subject requiring treatment. In certain embodiments, the antibody is an anti-CD20 antibody. In a more specific embodiment, the antibody is rituximab (e.g., RITUXAN®), ofatumumab (e.g., ARZERRA®), veltuzumab or ocrelizumab. In another embodiment, the antibody is an anti-tumor necrosis factor antibody. In more specific embodiments, the antibody is adalimumab (e.g., HUMIRA®), etanercept (e.g., ENBREL®), infliximab (e.g., REMICADE®), certolizumab pegol (e.g., CIMZIA®), natalizumab (e.g., TYSABRI®) or golimumab (e.g., SIMPONI®). In another specific embodiment, development of said oral lesion in said subject, wherein said subject is receiving or has received a course antibody therapy has caused a premature termination of said course of antibody therapy. In this context, “premature termination” means termination of the course of antibody therapy prior to what has been prescribed for said subject, partially or wholly as a result of said oral lesion.

4.3.4 Void Filling

In certain embodiments, the ECM compositions provided herein are used to seal, fill, and/or otherwise treat a void within the body of a subject. As used herein, the term “void” is intended to encompass any undesirable hollow space in a subject created by, e.g., aging, disease, surgery, congenital abnormalities, or a combination thereof. For example, a void may be created following the surgical removal of a tumor or other mass from the body of a subject. Non-limiting examples of voids which may be filled with the ECM compositions provided herein include a fissure, fistula, divercula, aneurysm, cyst, lesion, or any other undesirable hollow space in any organ or tissue of a subject's body.

In some embodiments, the ECM compositions provided herein may be used to fill, seal and/or otherwise treat, in whole or in part, a crevice, fissure, or fistula within a tissue, organ, or other structure of the body (e.g., a blood vessel), or junctures between adjacent tissues, organs or structures, to prevent the leakage of biological fluids, such as blood, urine, or other biological fluids. For example, the ECM compositions provided herein can be injected, implanted, threaded into, or otherwise administered into fistula between viscera, or into the opening or orifice from a viscus to the exterior of the subject's body. The ECM compositions provided herein can be used to fill a void or other defect formed by these pathological states and stimulate fibroblast infiltration, healing, and ingrowth of tissue.

In one embodiment, provided herein is a method to fill, seal, and/or otherwise treat a fistula in a subject, said method comprising injecting or otherwise administering to the subject an ECM composition provided herein. The ECM composition can be administered to the subject by injection through a needle into one of the fistular orifices and filling most or all of the branches of the orifice. Alternatively, strings or rods of the ECM composition can be threaded into the fistulae lesions through an orifice, or the collagen can be introduced into the subject with a catheter. Various types of fistulae can be filled, sealed and/or otherwise treated using the ECM compositions provided herein, such as anal, arteriovenous, bladder, carotid-cavernous, external, gastric, intestinal, parietal, salivary, vaginal, and anorectal fistulae, or a combination thereof.

In one embodiment, provided herein is a method to fill, seal and/or otherwise treat a diverticulum in a subject, said method comprising injecting or otherwise administering to the subject an ECM composition provided herein. Diverticulae are abnormal physiological structures that are pouches or sac openings from a tubular or saccular organ, such as the intestine, the bladder, and the like, and can be filled or augmented using the ECM compositions provided herein.

In another embodiment, provided herein is a method to fill, seal and/or otherwise treat a cyst in a subject, said method comprising injecting or otherwise administering to the subject an ECM composition provided herein. In some embodiments, the cyst is a pseudocyst, which has an accumulation of, e.g., fluid but does not comprise an epithelial or other membranous lining. Additional non-limiting examples of cysts that can be filled, sealed and/or otherwise treated include sebaceous, dermoid, bone, or serous cysts, or a combination thereof.

In another embodiment, the ECM compositions provided herein can be injected or otherwise administered to fill in whole, or in part, any void created as a result of surgical, chemical, or biological removal of unnecessary or undesirable growths, fluids, cells, or tissues from a subject. The ECM composition can be locally injected or otherwise administered at the site of the void so as to augment the remaining and surrounding tissue, aid in the healing process, and minimize the risk of infection. This augmentation is especially useful for void sites created after tumor excision, such as after breast cancer surgery, surgery for removal of tumorous connective tissue, bone tissues or cartilage tissue, and the like.

4.3.5 Tissue Bulking

In another embodiment, the ECM compositions provided herein can be used for tissue bulking. As used herein, “tissue bulking” refers to any change of the natural state of a subject's (e.g., a human's) non-dermal soft tissues due to external acts or effects. The tissues encompassed herein include, but not limited to, muscle tissues, connective tissues, fats, and, nerve tissues. The tissues may be part of many organs or body parts including, but not limited to, the sphincter, the bladder sphincter and urethra.

4.3.6 Urinary Incontinence

In another embodiment, the ECM compositions provided herein can be used for treatment of urinary incontinence (including stress urinary incontinence), which is the sudden leakage of urine that occurs with activities that result in an increase in intra-abdominal pressure, such as coughing, sneezing, laughing or exercise. In accordance with such embodiments, an ECM composition provided herein can be, e.g., injected into a subject so as to augment the subject's sphincter tissue, thereby improving or restoring in the subject. The ECM composition can be injected or otherwise administered periurethrally to increase tissue bulk around the urethra for the management and/or treatment of urinary incontinence. Improvement in stress incontinence can achieved by increasing the tissue bulk and thereby increasing resistance to the outflow of urine.

In some embodiments, the ECM composition is injected or otherwise administered to a subject in the area around the urethra, for example, to close a hole in the urethra through which urine leaks out or to build up the thickness of the wall of the urethra so it seals tightly when urine is being held back.

In another embodiment, the ECM composition is injected or otherwise administered to a subject around the urethra just outside the muscle of the urethra at the bladder outlet. Injecting the bulking material can be done through the skin, through the urethra, or, in women, through the vagina.

4.3.7 Vesicoureteral Reflux

In another embodiment, the ECM compositions provided herein can be used for treatment of vesicoureteral reflux (VUR) (or urinary reflux), which is characterized by the retrograde flow of urine from the bladder to the kidneys. In accordance with such embodiments, an ECM composition provided herein can be injected or otherwise administered to a subject in need thereof, wherein the ureteral wall of the subject is augmented, and the symptoms of VUR are reduced or eliminated. The composition can be injected (e.g., a subtrigonal injection) or otherwise administered, such as under endoscopic guidance, into the detrusor backing under the ureteral orifice using any method known to those in the art.

4.3.8 Gastroesophageal Reflux Disease

In another embodiment, the ECM compositions provided herein can be used for treatment of gastroesophageal reflux disease (GERD), which is a disorder that usually occurs because the lower esophageal sphincter (LES)—the muscular valve where the esophagus joins the stomach—does not close properly, relaxes or weakens, and stomach contents leak back, or reflux, into the esophagus. In accordance with such embodiments, an ECM composition provided herein can be injected or otherwise administered to a subject in need thereof, wherein the LES of the subject is augmented, and the symptoms of GERD are reduced or eliminated. In some embodiments, the ECM composition is administered under endoscopic guidance into the esophageal wall at the level of the esophagogastric junction. Intended to impede reflux, the bulking effect results from a combination of the retained material and consequent tissue response. The ECM composition can be injected through standard or large-bore (e.g., large gauge) injection needles.

4.3.9 Vocal Cords and Larynx

In another embodiment, the ECM compositions provided herein can be used in the management or treatment of a disease, disorder (such as a neurological disorder), or other abnormality that affects one or both vocal cords (folds) and/or the larynx (voice box). Non-limiting examples of such diseases, disorders or other abnormalities of the larynx and vocal cords include glottic incompetence, unilateral vocal cord paralysis, bilateral vocal cord paralysis, paralytic dysphonia, nonparalytic dysphonia, spasmodic dysphonia or a combination thereof. In other embodiments, the ECM compositions provided herein can be used to manage or treat diseases, disorders or other abnormalities that result in the vocal cords closing improperly, such as an incomplete paralysis of the vocal cord (“paresis”), generally weakened vocal cords, for instance, with old age (“presbylaryngis”), and/or scarring of the vocal cords (e.g., from previous surgery or radiotherapy).

The ECM compositions provided herein can be used to provide support or bulk to a vocal fold in a subject that lacks the bulk (such as in vocal fold bowing or atrophy) or the mobility (such as in paralysis) the vocal cord once had. In some embodiments, the vocal cords and/or other soft tissues of the larynx can be augmented with the ECM compositions provided herein, either alone or in combination with other treatments or medications. In one embodiment, an ECM composition provided herein augments or adds bulk to one (or both) vocal folds so that it can make contact with the other vocal fold.

Any one of a number of procedures well known to one of skill in the art can be used for administration of ECM compositions provided herein to a vocal cord(s) or larynx of a subject. In some embodiments, a curved needle is used to inject an ECM composition provided herein through the mouth of the subject. In other embodiments, a needle (such as a higher gauge, short needle) may be used to inject an ECM composition provided herein directly through the skin and the Adam's apple of the subject.

In certain embodiments, the ECM compositions provided herein can be used in the management or treatment of vocal cord paralysis. In one embodiment, the ECM compositions provided herein are used to manage or treat unilateral or bilateral vocal cord paralysis, or a symptom related thereto in a subject, by injecting or otherwise administering the ECM composition to the subject, wherein vocal fold closure is improved in the subject. In one embodiment, the ECM composition augments or adds bulk to one (or both) paralyzed vocal fold so that it can make contact with the other vocal fold. The injection of ECM composition to the subject can be through the subject's mouth or directly through the skin and Adam's apple.

In certain embodiments, the ECM compositions provided herein can be used to treat dysphonia, which is any impairment of the voice or difficulty speaking.

4.3.10 Glottic Incompetence

In another embodiment, the ECM compositions provided herein can be used for the management or treatment of glottic incompetence. Percutaneous laryngeal collagen augmentation can occur by injection of an ECM composition provided herein into the vocal cords of a subject using methods known in the art. In some cases, the subject has hypophonia and/or glottic incompetence that affects the voice function of the larynx, increased muscle rigidity, and decreased ability for movement of the thyroarytenoid muscle. In another embodiment, the hypophonia is a result of Parkinson's Disease. In one embodiment, the ECM composition can be used for the management or treatment of glottic incompetence in a subject in need thereof by injecting or otherwise administering the ECM composition to the vocal cords of the subject, wherein the injection augments the vocal cord and improves glottic closure, such that glottic incompetence is reduced or eliminated in the subject.

4.3.11 Bioengineering

In another embodiment, the ECM compositions provided herein can be used for bioengineering of tissue or organs, which can be used for, e.g., tissue replacement applications. Examples of bioengineered components that can be generated using the ECM compositions provided herein include, but are not limited to, bone, dental structures, joints, cartilage, skeletal muscle, smooth muscle, cardiac muscle, tendons, menisci, ligaments, blood vessels, stents, heart valves, corneas, ear drums, nerve guides, tissue or organ patches or sealants, a filler for missing tissues, sheets for cosmetic repairs, skin (sheets with cells added to make a skin equivalent), soft tissue structures of the throat such as trachea, epiglottis, and vocal cords, other cartilaginous structures such as nasal cartilage, tarsal plates, tracheal rings, thyroid cartilage, and arytenoid cartilage, connective tissue, vascular grafts and components thereof and sheets for topical applications, and repair to or replacement of organs such as livers, kidneys, and pancreas.

4.3.12 Cosmetic Applications

The ECM compositions provided herein are further useful in cosmetic applications. Generally, such cosmetic uses are based on the fact that the ECM compositions provided herein be used to fill in lines, creases, and other wrinkles in or on the skin of a subject and thus restore a smoother, more youthful-looking appearance.

In certain embodiments, the ECM compositions provided herein can be used for skin augmentation in a subject. In one embodiment, a method for skin augmentation in a subject comprises injecting or otherwise administering an ECM compositions provided herein to an area of the face or body of a subject in need of augmenting, wherein the area of the face or body of the subject is augmented as compared to the area prior to administration of the collagen. “Skin augmentation,” as used herein, refers to any change of the natural state of a subject's (e.g., a human's) skin and related areas due to external acts or effects. Non-limiting areas of the skin that may be changed by skin augmentation include the epidermis, dermis, subcutaneous layer, fat, arrector pill muscle, hair shaft, sweat pore, sebaceous gland, or a combination thereof.

Exemplary cosmetic uses of the ECM compositions provided herein include use of the ECM compositions to augment creased or sunken areas of the face and/or to add or increase the fullness to areas of the face and body of a subject; use of the ECM compositions to treat skin deficiencies including, but not limited to, wrinkles, depressions or other creases (e.g., frown lines, worry lines, crow's feet, marionette lines), stretch marks, internal and external scars (such as scars resulting from injury, wounds, accidents, bites, or surgery); use of the ECM compositions to correct “hollow” eyes and visible vessels resulting in dark circles around the eyes; use of the ECM compositions to correct or supplement plastic surgery, including correction of the undereye after aggressive removal of undereye fat pads from lower blepharoplasty, correction of the lower cheek after aggressive buccal fat extraction, and correction of the results of rhinoplasty, skin graft or other surgically-induced irregularities, such as indentations resulting from liposuction; use of the ECM compositions to correct facial or body scars (e.g., wound, chicken pox, or acne scars); and use of the ECM compositions for facial reshaping.

### 5. KITS

In another aspect, provided herein are kits comprising the ECM compositions provided herein.

In one embodiment, provided herein is a kit comprising an ECM composition provided herein, wherein said ECM composition is formulated as a sheet. The ECM sheet is provided in sterile form, and packaged in a pouch. The sheets of ECM provided in such kits can be of varying size (e.g., 5×5 cm or 9×9 cm) and thickness (e.g., 1.5-2.5 mm) and are ready for use by a practitioner, e.g., use as a wound dressing.

In another embodiment, provided herein is a kit comprising an ECM composition provided herein, wherein said ECM composition can be formulated as a flowable matrix. In such kits, an ECM composition is provided in sterile form, and packaged in a glass vial, as micronized ECM. Such vials can comprise varying amounts of micronized ECM, e.g., 200 mg of micronized ECM. Kits comprising an ECM composition that is to be formulated as a flowable matrix may further comprise components suitable for use in suspension of the micronized ECM, such as a suspension solution (e.g., saline) and a syringe with a needle and flexible applicator.

In another embodiment, provided herein is a kit comprising an ECM composition provided herein, wherein said ECM composition is formulated as a particulate. In such kits, an ECM composition is provided in sterile form, and packaged in a glass vial, as milled ECM. Such vials can comprise varying amounts of milled ECM, e.g., 100 mg or 200 mg of milled ECM.

The kits provided herein can comprise a label or labeling with instructions on using the ECM composition provided in the kit. In certain embodiments, the kits can comprise components useful for carrying out methods for which the ECM compositions are useful, such as means for administering the ECM composition in the kit, e.g., one or more spray bottles, tweezers, a spatula (for applying paste or particulate), cannulas, catheters, etc.

### 6. EXAMPLES

### 6.1. Example 1

### Method of Producing Placental ECM

This example describes methods of producing placental ECM, initially formulated as a paste.

Method 1: A previously isolated, frozen human placenta was obtained and allowed to thaw at room temperature for ˜24 hours. After the placenta was thawed, the amnion, chorion, and umbilical cord were removed from the placenta and discarded. Next, the placenta was cut into 2×2 centimeter strips for processing.

The placental tissue then was placed in receptacle containing 1.5 liters of a 1 M NaCl solution, and homogenized using an Omni Mixer Homogenizer (Omni International, Kennesaw, Ga.)). Next, the homogenized placental tissue was placed in a processing bag, and the bag was filled with a 1 M NaCL solution to a total volume of 9.2 liters. The homogenized placental tissue then was washed three times in a 1 M NaCl solution as follows: (i) the processing bag was agitated on an orbital shaker for 10 minutes, (ii) the placental tissue was allowed to settle in the processing bag for 10 minutes, and (iii) 6.2 liters of the supernatant was removed from the processing bag by gravity drainage, a step that removes blood and debris from the mixture.

After the third washing step, the washed placental tissue was allowed to mix in the 1 M NaCl solution (3 liters total of mixture) on an orbital shaker for ˜18-26 hours at room temperature. Next, the placental tissue was washed four times with sterile water, in the same manner described above for the NaCl washes. After the fourth wash in water, the placental tissue was allowed to mix in the water (3 liters total of mixture) on an orbital shaker for ˜18-26 hours at room temperature. After the ˜18-26 hour mixing in water, the placental tissue was washed a final time with water, as described above, then 6.2 L of 3% sodium deoxycholate was added to the mixture, for a final concentration of 2% sodium deoxycholate in the mixture.

The placental tissue was allowed to mix in the 2% sodium deoxycholate solution on an orbital shaker for ˜72 hours at room temperature. After the ˜72 hour mixing, the placental tissue was washed with sterile water five times, in the manner described above. After the final addition of water, the pH of the solution was brought to about 10-12 by dropwise addition of 1 M NaOH, resulting in a basic solution. The placental tissue was allowed to mix in the basic solution on an orbital shaker for ˜30 minutes at room temperature. After the ˜30 minutes of mixing, the pH of the solution was adjusted to about 7.0-7.5 by dropwise addition of 0.1 N HCl.

The supernatant then was removed from the processing bag and the placental tissue remaining was collected and centrifuged. After centrifugation, the supernatant was removed and the collected placental tissue was resuspended in sterile water, as a final wash step, and centrifuged again, followed by discarding of the supernatant. The resulting composition represented placental ECM comprising collagen and elastin, and was in the form of a white paste.

Method 2: Upon being released for processing, a frozen human placenta was thawed at 2-8° C. and then transferred to a biological safety cabinet (BSC), and then processed as done in Method 1. The placenta was removed from its storage container and placed on a sterile disposable tray. The placenta was then cleaned to remove excess blood and blood clots and then cut into small segments. The cut placental material was suspended in sterile water and then homogenized using a mechanical homogenizer, which generated small tissue particulates with increased surface area, allowing for more effective separation and removal of cells and cellular debris from placental ECM. The homogenized tissue from the placenta was transferred into a sterile processing bag with sterile 1 M sodium chloride solution. The tissue was washed several times with sterile 1 M sodium chloride (NaCl) by shaking on an orbital shaker; the NaCl solution was exchanged by allowing the placental tissue to settle, followed by draining and refreshing with additional sterile 1 M NaCl solution. The placental tissue was held for 18-24 hours with shaking in sterile 1 M NaCl solution, followed by repeated washing with sterile water. All processing steps were conducted at room temperature. The exposure of the placental tissue to a high concentration of sodium chloride, followed by water constitutes an “osmotic shock” to the tissue, which serves to clean the tissue of blood, blood components, cells and cellular debris. The placental tissue was subjected to a second “osmotic shock” before the next step, a detergent wash.

The rinsed placental tissue was held for 48-72 hours with sterile 0.1-0.3% sodium deoxycholate (DOC) solution and 4-8 mM ethylenediaminetetraacetic acid (EDTA) solution with shaking in the bio-processing bag at room temperature. Following a sterile water rinse, the tissue was subjected to a second wash with DOC/EDTA for 18-24 hours. Sterile water was then used to rinse the tissue and remove the DOC and EDTA.

Upon completion of the water wash, the supernatant was removed from the bio-processing bag and replaced with a solution of 2 mM magnesium chloride and 10 U/mL of BENZONASE®, pH 8-9, and mixed for 18-24 hours at room temperature. BENZONASE® is an endonuclease that degrades all forms of nucleic acids (RNA & DNA); the resulting shorter polynucleotide fragments are washed out with sequential rinses of the placental tissue.

After rinsing of the placental tissue to remove residual nucleic acids, the material was subjected to low and high pH treatments as viral inactivation steps. In the first step, the placental tissue was subjected to a pH of 3.3 or less in the presence of sterile 0.67 M NaCl solution and allowed to shake on an orbital shaker for 24 hours at 22 +/−1° C. In the second step, the pH of the solution was adjusted to ≧13 using sodium hydroxide (NaOH) and allowed to mix in the bio-processing bag, on an orbital shaker for a minimum of 4 hours at 22 +/−1° C. At the end of NaOH exposure, the pH of the solution was adjusted to a range of 5.5-9.0.

Upon completion of the acid and base treatments, the tissue was incubated with 1 M NaCl and allowed to mix on an orbital shaker for 48-72 hours. Following the NaCl treatment, the placental ECM was washed with sterile water for 18-24 hrs to remove debris and residual contaminants. ECM paste was generated by centrifuging the suspension. The ECM paste was stored in a −20° C. freezer until the product was formulated and sterilized.

Formulation as sheet: To generate ECM sheets, the ECM paste was thawed at 2° C. to 22° C. for 24-48 hours, and resuspended in sterile phosphate buffer in a biological safety cabinet. The tissue was homogenized to prepare a homogenous ECM suspension, which was distributed into sterile plastic molds and frozen. The frozen molds of ECM were dehydrated using lyophilization. The resulting dehydrated ECM wafers were re-hydrated with sterile water and then compacted on a vacuum-assisted dryer for 18-36 hours at room temperature. Sheets were placed into a double pouch and sealed using a medical grade sealer, labeled, and sterilized using gamma radiation.

Formulation as a particulate: To generate ECM particulate, the ECM paste is resuspended in sterile water, transferred into molds, frozen using a Controlled Rate Freezer (Thermo Scientific, Marietta, Ohio) and lyophilized in a freeze-dryer (LabConco, Kansas City, Mo.) for 48 hours. The lyophilized ECM is milled using a jet mill (Fluid Energy, Telford, Pa.), resulting in ECM particulate. The milled ECM powder can be filled into amber glass vials and sealed.

Formulation as flowable matrix: ECM flowable matrix can be prepared using ECM particulate. Generally, ECM flowable matrix can be prepared by suspending lyophilized, milled ECM (ECM particulate in, e.g., sterile water or saline solution.

### 6.2. Example 2

### Determination of Collagen Content of ECM Composition

This Example explains the analysis of collagen content of the ECM composition.

ECM composition, prepared as described in Example 1 in particulate, sheet or flowable matrix form, was analyzed for total collagen content using a colorimetric hydroxyproline assay. Collagen is a unique protein in that it has a very high proline concentration. In addition, collagen is post-translationally modified to hydroxyproline. As such, quantification of hydroxyproline, which can be obtained from collagen hydrolyzed in hot hydrochloric acid can be used as a surrogate for quantification of collagen.

ECM samples were hydrolyzed using 6 N HCl at 110° C., and hydroxyproline was then oxidized to pyrrole-2-carboxylic acid (pyrrole) using Chloramine-T (N-chloro 4-methylbenzenesulfonamide). Color was developed using 4-Dimethylaminobenzaldehyde (DMAB), and hydroxyproline content determined by reading the absorbance at 550 nm. Sample absorbances were interpolated against a hydroxyproline standard curve using known amounts of hydroxyproline.

The ECM composition was determined to comprise a range of 31% total collagen by weight to 53% total collagen by weight of the ECM composition (Table 1). 14/16 samples fell within a range of 34% to 43% total collagen by weight of the ECM composition, and 12/16 fell within a range of 37% to 42% total collagen by weight of the ECM composition. In contrast, comparator matrix products derived from porcine urinary bladder matrix (MATRISTEM® Wound Matrix sheets or MATRISTEM MICROMATRIX® particulates) and fetal bovine dermis matrix (PRIMATRIX™ Dermal Repair Matrix) were found to comprise 67%, 67%, and 69% total collagen by weight, respectively.

TABLE 1
Total collagen as a percent by dry weight of ECM composition

| Sample | Type | Collagen % by weight |
|---|---|---|
| 1 | Particulate | 37 |
| 2 | Sheet | 43 |
| 3 | Particulate | 42 |
| 4 | Particulate | 34 |
| 5 | Sheet | 41 |
| 6 | Particulate | 38 |
| 7 | Particulate | 38 |
| 8 | Flowable Matrix | 42 |
| 9 | Sheet | 38 |
| 10 | Particulate | 53 |
| 11 | Particulate | 40 |
| 12 | Particulate | 42 |
| 13 | Particulate | 38 |
| 14 | Particulate | 37 |
| 15 | Particulate | 42 |
| 16 | Particulate | 31 |
| Porcine urinary bladder matrix | Sheet | 67 |
| Porcine urinary bladder matrix | Particulate | 67 |
| Fetal bovine dermal matrix | Sheet | 69 |

### 6.3. Example 3

### Determination of Elastin Content of ECM Composition

This Example explains the analysis of elastin content of the ECM composition.

ECM composition, prepared as described in Example 1 as either particulate or sheets, was analyzed for elastin content using the Fastin Elastin Assay kit (BioColor, Ltd. (UK)), which uses 5, 10, 15, 20-tetraphenyl-21, 23-porphine tetra-sulfonate (TPPS) as a dye to stain extracted and solubilized α-elastin from test samples. Elastin was extracted from ECM composition samples in 0.25 M oxalic acid at 100° C. Elastin was then precipitated with trichloroacetic acid and hydrochloric acid (TCA/HCl), and then stained with TPPS. The TPPS was then dissociated from the elastin, and released TPPS levels were quantified by spectrophotometry. Elastin levels in the ECM composition samples were determined by interpolation against an elastin standard curve.

The ECM composition was determined to have a range of 13% to 29% elastin by weight of the ECM composition (Table 2). 14/16 samples fell within a range of 16% to 24% elastin by weight of the ECM composition, 13/16 samples fell within a range of 17% to 24% elastin by weight of the ECM composition, and 10/16 samples fell within a range of 20% to 24% elastin by weight of the ECM composition. In contrast, the comparator matrix products derived from porcine urinary bladder matrix (MATRISTEM® Wound Matrix sheets or MATRISTEM MICROMATRIX® particulates) were found to comprise 4% elastin. Elastin was not detected in fetal bovine dermis matrix (PRIMATRIX™ Dermal Repair Matrix).

In Tables 2 and 3, sample 8 represents the same ECM composition as sample 8 in Table 1. However, the type of ECM composition analyzed differed (i.e., the flowable matrix form of the ECM composition was analyzed in Table 1, whereas the particulate form of the ECM composition was analyzed in tables 2 and 3).

TABLE 2
Elastin percentage of total ECM composition by dry weight

| Sample | Type | Elastin % by Weight |
|---|---|---|
| 1 | Particulate | 17 |
| 2 | Sheet | 20 |
| 3 | Particulate | 17 |
| 4 | Particulate | 16 |
| 5 | Sheet | 24 |
| 6 | Particulate | 24 |
| 7 | Particulate | 20 |
| 8 | Particulate | 21 |
| 9 | Sheet | 22 |
| 10 | Particulate | 13 |
| 11 | Particulate | 17 |
| 12 | Particulate | 22 |
| 13 | Particulate | 20 |
| 14 | Particulate | 29 |
| 15 | Particulate | 25 |
| 16 | Particulate | 23 |
| Average: | | 21 |
| MatriStem Wound Matrix | Sheet | 4 |
| Matristem Micromatrix | Particulate | 4 |
| Primatrix Sheet | Sheet | Not detected |

### 6.4. Example 4

### Determination of Fibronectin Content of ECM Composition

This Example describes the analysis of fibronectin content of the ECM composition.

ECM composition, prepared as described in Example 1 as either particulate or sheets, was analyzed for fibronectin content using the TaKaRa Fibronectin EIA kit (Mountain View, Calif.), which is based on a sandwich ELISA method that utilizes two mouse monoclonal anti-human fibronectin antibodies to detect fibronectin by a two-step procedure. Additionally, a specific extraction method for fibronectin using 2 M urea buffer was developed.

15 mg of ECM composition was mixed with 3 mL of extraction buffer (2 M urea, 0.05 M PO4, 2 mM PMSF, pH 7.1) and mixed on a stir plate for 4 hours. The sample was then centrifuged for 20 minutes at 10,000×g. Supernatants were stored at −20° C. until the time of analysis, or used immediately. Analysis of 16 ECM composition samples using the TaKaRa Fibronectin EAI Kit was performed according to manufacturer's instructions. A standard curve of fibronectin concentrations was constructed using commercially-available lyophilized fibronectin, and sample fibronectin concentrations were interpolated from the curve.

Fibronectin content of the ECM composition was low, with an average of 197 ng of fibronectin per mg of ECM composition (0.0197% by weight of the ECM composition), and a range of 21.3 ng/mg to 361.3 ng/mg. Samples of porcine urinary bladder matrix (MATRISTEM® Wound Matrix sheets or MATRISTEM MICROMATRIX® particulates) and fetal bovine dermis matrix (PRIMATRIX™ Dermal Repair Matrix) were tested but revealed no fibronectin content in the assay, possibly because the antibodies used in the assay did not cross-react with the bovine or porcine matrix.

TABLE 3
Fibronectin percentage of total ECM composition by dry weight

| Sample | Type | Average ng/mg |
|---|---|---|
| 1 | Particulate | 332.7 |
| 2 | Sheet | 181.0 |
| 3 | Particulate | 114.8 |
| 4 | Particulate | 270.0 |
| 5 | Sheet | 21.3 |
| 6 | Particulate | 194.1 |
| 7 | Particulate | 112.6 |
| 8 | Particulate | 150.1 |
| 9 | Sheet | 71.8 |
| 10 | Particulate | 138.1 |
| 11 | Particulate | 97.0 |
| 12 | Particulate | 200.7 |
| 13 | Particulate | 217.0 |
| 14 | Particulate | 361.3 |
| 15 | Particulate | 342.8 |
| 16 | Particulate | 346.8 |
| Average | | 197.0 |
| MatriStem MicroMatrix | Particulate | Not detected |
| MatriStem Wound Matrix | Sheet | Not detected |
| PriMatrix | Sheet | Not detected |

### 6.5. Example 5

### Determination of Laminin Composition of ECM Composition

Sixteen samples of ECM composition, prepared as described in Example 1 and formulated as a sheet or particulate, were analyzed for laminin content using the TaKaRa Laminin EIA kit (Mountain View, Calif.), according to manufacturer instructions. The kit is based on a sandwich ELISA method that utilizes two mouse monoclonal anti-human laminin antibodies to detect laminin by a two-step procedure. Additionally, a specific extraction method for laminin using 2 M urea buffer was developed. A standard curve of laminin concentrations was constructed using commercially-available lyophilized laminin, and sample laminin concentrations were interpolated from the curve.

Results: Laminin was not detected in any of the 16 ECM samples. Laminin also was not detected in any of the porcine urinary bladder matrix (sheet or particulate) or fetal bovine dermis sheets analyzed for comparison.

### 6.6. Example 6

### Biocompatibility of ECM Composition

ECM composition, prepared as described in Example 1 and formulated as a sheet, a particulate, or a flowable matrix, was tested for biocompatibility in albino New Zealand White Rabbits, 4.7-6.3 months in age and weighing 2.4-2.9 kg at study start. As controls, ECM sheet was compared to MATRISTEM® Wound Matrix (porcine urinary bladder matrix); ECM particulate was compared to MATRISTEM MICROMATRIX®, and ECM flowable matrix was compared to INTEGRA™ Flowable Wound Matrix (collagen and glycosaminoglycan).

| Test Article | Amount Used/Site | |
|---|---|---|
| ECM Particulate | 5.0-5.4 | mg |
| ECM Flowable | 50 | μL |
| ECM Sheet | 1 × 3 × 10 | mm pieces |
| MATRISTEM ® Particulate | 5.0-5.4 | mg |
| Integra Flowable | 50 | μL |
| MatriStem Sheet | 1 × 3 × 10 | mm pieces |

Preparation of flowable matrix: ECM Flowable and INTEGRA™ Flowable were prepared by aspirating over 2 mL of injectable water with a 3 mL Vial Access syringe, eliminating any air bubbles, and adjusting the volume to 2 mL. The cannula was then removed from the Vial Access syringe. This syringe was then connected to an ECM syringe containing 2 mL water tip-to-tip. The water was slowly injected from the vial access syringe into the Vial Access syringe, and carefully mixed using 10 to 15 back and forth movements until the powder was homogenously hydrated. The entire reconstituted paste was pushed into one of the syringes. The empty syringe was then discarded, an empty 1 mL syringe was connected to the remaining ECM paste syringe, and 1 mL of the ECM Flowable or INTEGRA™ Flowable paste was transferred into the 1 mL syringe. This last step was repeated with a second 1 mL syringe. The pastes were re-mixed prior to loading into the 1 mL syringe for each dose. INTEGRA™ Flowable was mixed with 3.0 mL of saline for injection. The test and control articles were applied directly using the filled 1 mL syringes.

Implantation: For each animal a 2-4 cm skin incision was made over the midline of the back, extending through the fascia of both paravertebral muscles. For each implant site, a 5 mm incision was made into the paravertebral muscle and a small pocket was generated with hemostats for implantation, approximately 2 cm from the midline and parallel to the muscle fiber axis, allowing at least 2.5 cm between implant sites. Four test or four control articles were implanted in one side of the paravertebral muscle. The test article was implanted on the right side and the control article on the left side for each group. All implant sites were then closed with non-absorbable suture, and the skin incision was closed with suture and/or skin staple. 50 μL of the flowable formulation, 5 mg of particulate formulation and 1 piece (1×3×10 mm) of sheet formulation were implanted into the generated pocket of the muscle. Control articles were 50 μL of bovine derived wound matrix product (INTEGRA™ Flowable), 5 mg of particulate formulation of extra cellular matrix derived from porcine urinary bladder (MATRISTEM MICROMATRIX®), and a sheet formulation (1×3×10 mm) derived from porcine urinary bladder extra cellular matrix (MATRISTEM WOUND MATRIX®) Animals were killed at weeks 1, 2 or 4 post-implantation, and histology was performed on the

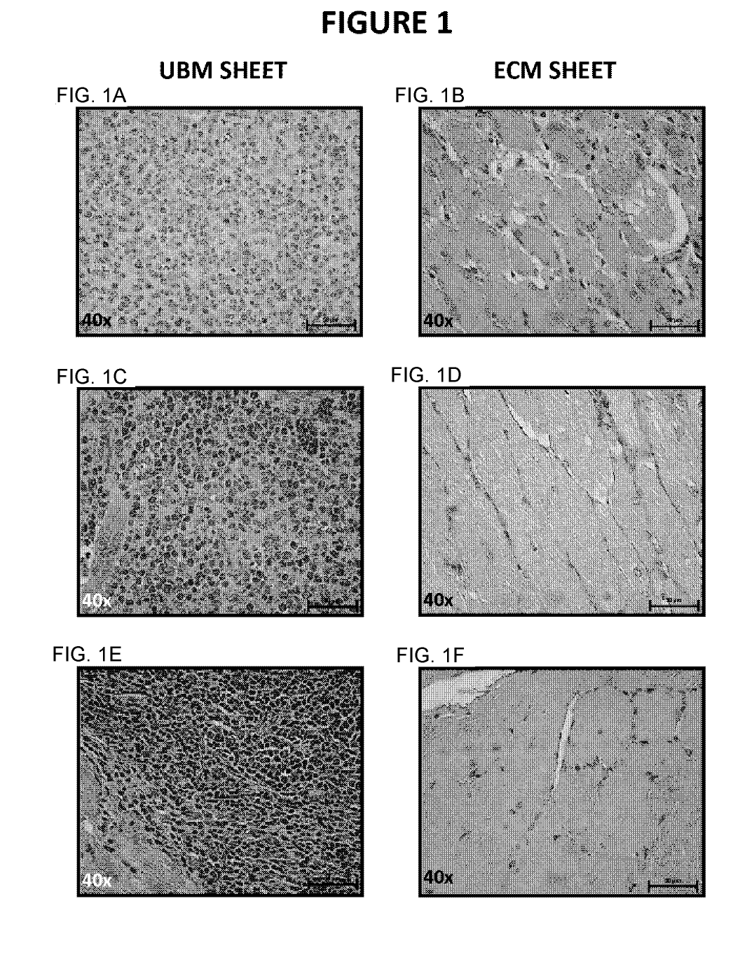

Results: ECM sheet showed demonstrably less tissue reactivity than did the porcine urinary bladder matrix. At week 1 postimplantation, the tissue adjacent to the urinary bladder matrix (UBM) sheet showed distinct signs of granulation and inflammatory response (FIG. 1A), while the ECM sheet showed muscle tissue interspersed with slight infiltration of granulocytes. At weeks 2 and 4, granulation was still in evidence adjacent to the UBM sheet (FIGS. 1C and 1E), while tissue adjacent to the ECM sheet showed virtually no granulation and appeared to be normal (FIGS. 1D and 1F). The ECM sheet was deemed to be a non-irritant at weeks 1, 2 and 4 post-implantation.

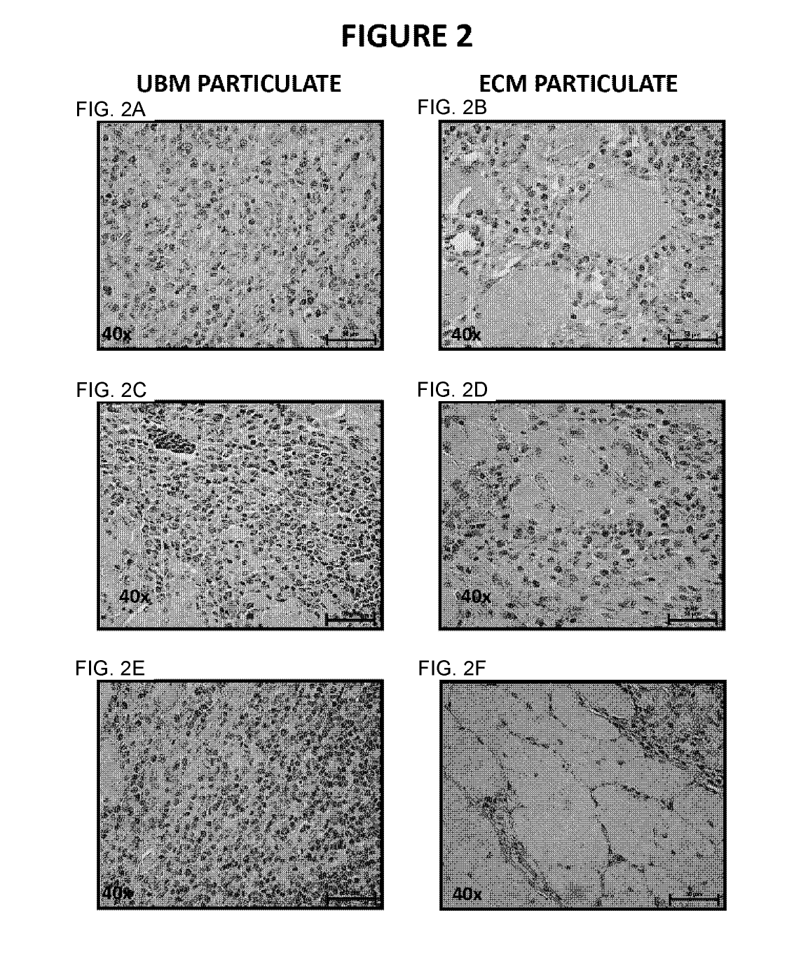

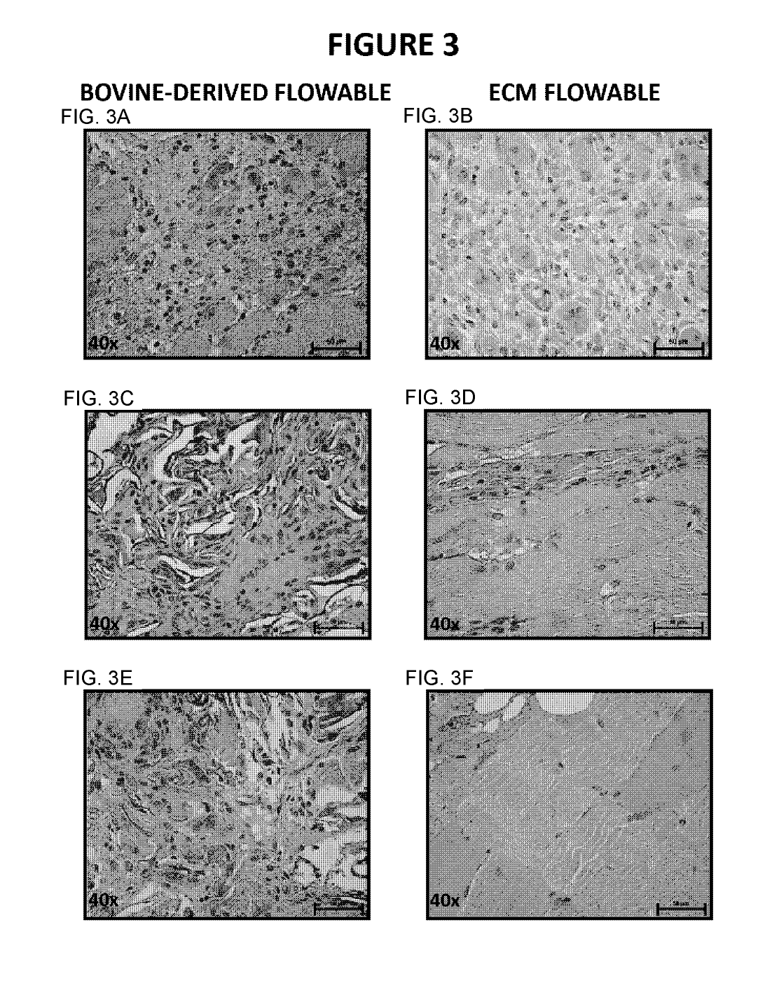

ECM particulate and flowable matrix also engendered less tissue reactivity than the control particulate or flowable matrix (FIGS. 2 and 3). For example, at week 1 postimplantation, the ECM particulate showed some granulation indicating inflammation (FIG. 2B), but significantly less than the UBM particulate (FIG. 2A), while at weeks 2 and 4, the ECM particulate showed a significant reduction of granulation (FIGS. 2D and 2F, respectively) as compared to the UBM particulate, which still showed substantial inflammation at weeks 2 and 4 (FIGS. 2C and 2E, respectively). Similarly, while the bovine derived wound matrix product (INTEGRA™ Flowable) showed granulation at week 1 (FIG. 3A), followed by scarring at weeks 2 and 4 (lighter areas in FIGS. 3C and 3E), the ECM flowable showed an inflammatory response substantially only in the first week (FIG. 3B), followed by near-complete healing at weeks 2 and 4 (FIGS. 3D and 3F, respectively).

### 6.7. Example 7

### Method of Producing Placental ECM from Placental Chorion

This example describes methods of producing placental ECM from placental chorion, initially formulated as a paste.

Method 1: A previously isolated, frozen human placenta is obtained and allowed to thaw at room temperature for ˜24 hours. After the placenta is thawed, the chorion is obtained from the placenta. Next, the placenta is cut into 2×2 centimeter strips for processing.

The placental chorion tissue then is placed in a receptacle containing 1.5 liters of a 1 M NaCl solution, and homogenized using an Omni Mixer Homogenizer (Omni International, Kennesaw, Ga.)). Next, the homogenized placental chorion tissue is placed in a processing bag, and the bag is filled with a 1 M NaCL solution to a total volume of 9.2 liters. The homogenized placental chorion tissue then is washed three times in a 1 M NaCl solution as follows: (i) the processing bag is agitated on an orbital shaker for 10 minutes, (ii) the placental chorion tissue is allowed to settle in the processing bag for 10 minutes, and (iii) 6.2 liters of the supernatant is removed from the processing bag by gravity drainage, a step that removes blood and debris from the mixture.

After the third washing step, the washed placental chorion tissue is allowed to mix in the 1 M NaCl solution (3 liters total of mixture) on an orbital shaker for ˜18-26 hours at room temperature. Next, the placental tissue is washed four times with sterile water, in the same manner described above for the NaCl washes. After the fourth wash in water, the placental chorion tissue is allowed to mix in the water (3 liters total of mixture) on an orbital shaker for ˜18-26 hours at room temperature. After the ˜18-26 hour mixing in water, the placental chorion tissue is washed a final time with water, as described above, then 6.2 L of 3% sodium deoxycholate is added to the mixture, for a final concentration of 2% sodium deoxycholate in the mixture.

The placental chorion tissue is allowed to mix in the 2% sodium deoxycholate solution on an orbital shaker for ˜72 hours at room temperature. After the ˜72 hour mixing, the placental chorion tissue is washed with sterile water five times, in the manner described above. After the final addition of water, the pH of the solution is brought to about 10-12 by dropwise addition of 1 M NaOH, resulting in a basic solution. The placental chorion tissue is allowed to mix in the basic solution on an orbital shaker for ˜30 minutes at room temperature. After the ˜30 minutes of mixing, the pH of the solution is adjusted to about 7.0-7.5 by dropwise addition of 0.1 N HCl.

The supernatant then is removed from the processing bag and the placental chorion tissue remaining is collected and centrifuged. After centrifugation, the supernatant is removed and the collected placental chorion tissue is resuspended in sterile water, as a final wash step, and centrifuged again, followed by discarding of the supernatant. The resulting composition represents placental ECM comprising collagen and elastin, and will be in the form of a white paste.

Method 2: Upon being released for processing, a frozen human placenta is thawed at 2-8° C. and then transferred to a biological safety cabinet (BSC). The placenta is removed from its storage container and placed on a sterile disposable tray. The placenta is then cleaned to remove excess blood and blood clots. The chorion of the placenta is obtained and cut into small segments. The cut placental chorion material is suspended in sterile water and then homogenized using a mechanical homogenizer, which will generate small tissue particulates with increased surface area, allowing for more effective separation and removal of cells and cellular debris from placental ECM. The homogenized tissue from the placental chorion is transferred into a sterile processing bag with sterile 1 M sodium chloride solution. The tissue is washed several times with sterile 1 M sodium chloride (NaCl) by shaking on an orbital shaker; the NaCl solution is exchanged by allowing the placental tissue to settle, followed by draining and refreshing with additional sterile 1 M NaCl solution. The placental tissue is held for 18-24 hours with shaking in sterile 1 M NaCl solution, followed by repeated washing with sterile water. All processing steps are conducted at room temperature. The exposure of the placental chorion tissue to a high concentration of sodium chloride, followed by water constitutes an “osmotic shock” to the tissue, which serves to clean the tissue of blood, blood components, cells and cellular debris. The placental tissue is subjected to a second “osmotic shock” before the next step, a detergent wash.

The rinsed placental chorion tissue is held for 48-72 hours with sterile 0.1-0.3% sodium deoxycholate (DOC) solution and 4-8 mM ethylenediaminetetraacetic acid (EDTA) solution with shaking in the bio-processing bag at room temperature. Following a sterile water rinse, the tissue is subjected to a second wash with DOC/EDTA for 18-24 hours. Sterile water is then used to rinse the tissue and remove the DOC and EDTA.

Upon completion of the water wash, the supernatant is removed from the bio-processing bag and replaced with a solution of 2 mM magnesium chloride and 10 U/mL of BENZONASE®, pH 8-9, and mixed for 18-24 hours at room temperature. BENZONASE® is an endonuclease that degrades all forms of nucleic acids (RNA & DNA); the resulting shorter polynucleotide fragments are washed out with sequential rinses of the placental tissue.

After rinsing of the placental chorion tissue to remove residual nucleic acids, the material is subjected to low and high pH treatments as viral inactivation steps. In the first step, the placental chorion tissue is subjected to a pH of 3.3 or less in the presence of sterile 0.67 M NaCl solution and allowed to shake on an orbital shaker for 24 hours at 22 +/−1° C. In the second step, the pH of the solution is adjusted to ≧13 using sodium hydroxide (NaOH) and allowed to mix in the bio-processing bag, on an orbital shaker for a minimum of 4 hours at 22 +/−1° C. At the end of NaOH exposure, the pH of the solution is adjusted to a range of 5.5-9.0.

Upon completion of the acid and base treatments, the tissue is incubated with 1 M NaCl and allowed to mix on an orbital shaker for 48-72 hours. Following the NaCl treatment, the placental chorion ECM is washed with sterile water for 18-24 hrs to remove debris and residual contaminants. ECM paste is generated by centrifuging the suspension. The ECM paste can be stored in a −20° C. freezer until the product is formulated and sterilized.

Formulation as sheet: To generate ECM sheets, the ECM paste is thawed at 2° C. to 22° C. for 24-48 hours, and resuspended in sterile phosphate buffer in a biological safety cabinet. The tissue is homogenized to prepare a homogenous ECM suspension, which is distributed into sterile plastic molds and frozen. The frozen molds of ECM are dehydrated using lyophilization. The resulting dehydrated ECM wafers are re-hydrated with sterile water and then compacted on a vacuum-assisted dryer for 18-36 hours at room temperature. Sheets are placed into a double pouch and sealed using a medical grade sealer, labeled, and sterilized using gamma radiation.

Formulation as a particulate: To generate ECM particulate, the ECM paste is resuspended in sterile water, transferred into molds, frozen using a Controlled Rate Freezer (Thermo Scientific, Marietta, Ohio) and lyophilized in a freeze-dryer (LabConco, Kansas City, Mo.) for 48 hours. The lyophilized ECM is milled using a jet mill (Fluid Energy, Telford, Pa.), resulting in ECM particulate. The milled ECM powder can be filled into amber glass vials and sealed.

Formulation as flowable matrix: ECM flowable matrix can be prepared using ECM particulate. Generally, ECM flowable matrix can be prepared by suspending lyophilized, milled ECM (ECM particulate in, e.g., sterile water or saline solution.

The collagen, elastin, fibronectin, and/or laminin content of the ECM can be analyzed in the manner described in Examples 2, 3, 4, and 5, respectively. Further, biocompatibility of the ECM can be assessed in the manner described in Example 6.

### 6.8. Example 8

### Method of Producing Placental ECM from Placental Chorion (2)

This example describes a method for producing a placental ECM composition from placental chorion, initially formulated as a paste.

A human placenta obtained from a mother immediately after a full-term birth, or a previously isolated frozen human placenta that has been allowed to thaw, is utilized. The placenta is washed in 0.5 M NaCl. The amnion, umbilical cord and decidua parietalis is removed from the placenta, and the chorionic plate is retained, which is then scraped and cleaned. The scraped, cleaned chorion is rinsed in 0.5 M NaCl and water, and then rinsed overnight in 2% deoxycholic acid, followed by several water rinses. The treated chorion is then ground and freeze dried. The resulting composition is a decellularized ECM paste suitable for further formulation, e.g., milling and formulation.

Formulation as sheet: To generate ECM sheets, the ECM paste is thawed at 2° C. to 22° C. for 24-48 hours, and resuspended in sterile phosphate buffer in a biological safety cabinet. The tissue is homogenized to prepare a homogenous ECM suspension, which is distributed into sterile plastic molds and frozen. The frozen molds of ECM are dehydrated using lyophilization. The resulting dehydrated ECM wafers are re-hydrated with sterile water and then compacted on a vacuum-assisted dryer for 18-36 hours at room temperature. Sheets are placed into a double pouch and sealed using a medical grade sealer, labeled, and sterilized using gamma radiation.

Formulation as a particulate: To generate ECM particulate, the ECM paste is resuspended in sterile water, transferred into molds, frozen using a Controlled Rate Freezer (Thermo Scientific, Marietta, Ohio) and lyophilized in a freeze-dryer (LabConco, Kansas City, Mo.) for 48 hours. The lyophilized ECM is milled using a jet mill (Fluid Energy, Telford, Pa.), resulting in ECM particulate. The milled ECM powder can be filled into amber glass vials and sealed.

Formulation as flowable matrix: ECM flowable matrix can be prepared using ECM particulate. Generally, ECM flowable matrix can be prepared by suspending lyophilized, milled ECM (ECM particulate in, e.g., sterile water or saline solution.

The collagen, elastin, fibronectin, and/or laminin content of the ECM can be analyzed in the manner described in Examples 2, 3, 4, and 5, respectively. Further, biocompatibility of the ECM can be assessed in the manner described in Example 6.

### 6.9. Example 9

### Method of Producing Placental ECM from Placental Chorion (3)

This example describes a method for producing a placental ECM composition from placental chorion.

A human placenta obtained from a mother immediately after a full-term birth is utilized. The amnion, umbilical cord and decidua parietalis is removed from the placenta, and the chorionic plate is retained, which is then scraped and cleaned. The scraped, cleaned chorion is rinsed in 1.0 M NaCl and water, and then rinsed overnight in a solution containing 0.067% deoxycholic acid and 4 mM EDTA, followed by several water rinses. The chorion is then rinsed overnight in a solution containing 0.39% deoxycholic acid and 8 mM EDTA, followed by several water rinses. The treated whole chorion is then freeze dried.

Formulation as sheet: To generate ECM sheets, the ECM is thawed at 2° C. to 22° C. for 24-48 hours, and resuspended in sterile phosphate buffer in a biological safety cabinet to form an ECM paste. The tissue is homogenized to prepare a homogenous ECM suspension, which is distributed into sterile plastic molds and frozen. The frozen molds of ECM are dehydrated using lyophilization. The resulting dehydrated ECM wafers are re-hydrated with sterile water and then compacted on a vacuum-assisted dryer for 18-36 hours at room temperature. Sheets are placed into a double pouch and sealed using a medical grade sealer, labeled, and sterilized using gamma radiation.

Formulation as a particulate: To generate ECM particulate, the ECM is resuspended in sterile water to form an ECM paste, transferred into molds, frozen using a Controlled Rate Freezer (Thermo Scientific, Marietta, Ohio) and lyophilized in a freeze-dryer (LabConco, Kansas City, Mo.) for 48 hours. The lyophilized ECM is milled using a jet mill (Fluid Energy, Telford, Pa.), resulting in ECM particulate. The milled ECM powder can be filled into amber glass vials and sealed.

Formulation as flowable matrix: ECM flowable matrix can be prepared using ECM particulate. Generally, ECM flowable matrix can be prepared by suspending lyophilized, milled ECM (ECM particulate in, e.g., sterile water or saline solution.

The collagen, elastin, fibronectin, and/or laminin content of the ECM can be analyzed in the manner described in Examples 2, 3, 4, and 5, respectively. Further, biocompatibility of the ECM can be assessed in the manner described in Example 6.

Characterization of ECM compositions, pre-sterilization, produced using such a method exhibited the dry weight characteristics summarized in Table 4, below:

TABLE 4

| Collagen | 43-68% | |
|---|---|---|
| Elastin | 18-21% | |
| Fibronectin | 27-5322 | ng/mg (<0.05%) |
| Laminin | 28-568 | ng/ml (<0.05%) |
| Glycosaminoglycans | 0.2-1.2 | μg/mg (<0.05%) |

| | |
|---|---|
| Cytokines | <80 pg/mL (below level of detection) |
| Growth Factors | <80 pg/mL (below level of detection) |
| Deoxycholic Acid | <300 parts per million (below level of detection) |
| Cell-free | >90% |
| Cellular debris-free | >90% |

All publications and patent applications cited in this specification are herein incorporated by reference as if each individual publication or patent application were specifically and individually indicated to be incorporated by reference. Although the foregoing has been described in some detail by way of illustration and example for purposes of clarity of understanding, it will be readily apparent to those of ordinary skill in the art in light of the teachings provided herein that certain changes and modifications may be made thereto without departing from the spirit or scope of the appended claims.

1. An extracellular matrix (ECM) composition comprising collagen and elastin prepared from a chorionic plate that has been separated from a placenta.

2. The composition of claim 1, wherein said composition comprises less than 0.1% fibronectin.

3. The composition of claim 1, wherein said composition comprises an undetectable amount of laminin.

4. The composition of claim 1, wherein said composition comprises an undetectable amount of glycosaminoglycans.

5. The composition of claim 1, wherein said composition comprises less than 0.1% fibronectin, an undetectable amount of laminin, and an undetectable amount of glycosaminoglycans.

6. The composition of claim 1, wherein the composition comprises about 30-72% collagen and about 10-30% elastin.

7. The composition of claim 6, wherein said composition comprises less than about 0.1% fibronectin.

8. The composition of claim 6, wherein said composition comprises less than about 0.1% laminin.

9. The composition of claim 6, wherein said composition comprises less than about 0.1% glycosaminoglycans.

10. The composition of claim 6, wherein said composition comprises an undetectable amount of cytokines and growth factors.

11. The composition of claim 6, wherein said composition comprises less than about 0.1% fibronectin, less than about 0.1% laminin, less than about 0.1% glycosaminoglycans, and an undetectable amount of cytokines and growth factors.

12-13. (canceled)

14. The composition of claim 11, wherein said placenta is a human placenta.

15. The composition of claim 1, wherein said composition is formulated as a sheet.

16. (canceled)

17. The composition of claim 1, wherein said composition is formulated as a particulate.

18. The composition of claim 1, wherein said composition is formulated as a flowable matrix.

19-55. (canceled)

56. The composition of claim 1, wherein the composition comprises about 30-72% collagen.

57. The composition of claim 1, wherein the composition comprises at least about 60% type I collagen.

58. The composition of claim 1, wherein the composition comprises about 10-35% elastin.

59. The composition of claim 58, wherein the composition comprises about 10-20% elastin.

60. The composition of claim 1, wherein the composition is decellularized.

61. The composition of claim 17, wherein the particulate is packaged in a suitable container.

62. The composition of claim 61, wherein the container contains about 200-300 mg, 100-200 mg, 150-250 mg, 50-100 mg, 25-50 mg, 10-25 mg, 5-10 mg, or 1-5 mg of the particulate.

63. The composition of claim 61, further comprising a pharmaceutically or cosmetically acceptable carrier that is added to the particulate prior to use.

64. The composition of claim 18, wherein the flowable matrix comprises 200-300 mg/ml, 100-200 mg/ml, 150-250 mg/ml, 0.1-100 mg/ml, 1-75 mg/ml, 1-50 mg/ml, 1-40 mg/ml, 10-40 mg/ml, or 20-40 mg/ml of ECM.

65. The composition of claim 61, wherein the container is a syringe.

66. The composition of claim 15, wherein the composition comprises a laminate comprising two or more sheets.

67. The composition of claim 66, wherein the laminate comprises one or more ECM sheets and a sheet that comprises a different planar decellularized tissue or a planar artificial tissue substitute.

68. The composition of claim 66, wherein the laminate comprises one or more ECM sheets.
